SEQUENCE LISTING (43 sequences)

SEQ ID NO: 1 - DNA, 592 bp, Homo sapiens
atgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggct

SEQ ID NO: 2 - DNA, 603 bp, Homo sapiens
atggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccg

SEQ ID NO: 3 - DNA, 600 bp, Homo sapiens
ggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacaggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatt

SEQ ID NO: 4 - DNA, 22 bp, Artificial Sequence
  Description of Artificial Sequence Synthetic primer
cgggtctcaaactgcccagctt

SEQ ID NO: 5 - DNA, 19 bp, Artificial Sequence
  Description of Artificial Sequence Synthetic primer
ggctgggtcactggccctc

SEQ ID NO: 6 - DNA, 18 bp, Artificial Sequence
  Description of Artificial Sequence Synthetic primer
acctccaccatgccaagt

SEQ ID NO: 7 - DNA, 18 bp, Artificial Sequence
  Description of Artificial Sequence Synthetic primer
tagttcccgaaaccctga

SEQ ID NO: 8 - DNA, 25 bp, Artificial Sequence
  Description of Artificial Sequence Synthetic primer
agtcggagatggcccagatacaaca

SEQ ID NO: 9 - DNA, 25 bp, Artificial Sequence
  Description of Artificial Sequence Synthetic primer
tccagcagttggatttcaagacggg

SEQ ID NO: 10 - DNA, 25 bp, Artificial Sequence
  Description of Artificial Sequence Synthetic primer
tacgtgctgaagatccagctgaagg

SEQ ID NO: 11 - DNA, 25 bp, Artificial Sequence
  Description of Artificial Sequence Synthetic primer
agttggaaggaccacatgcgtcgaa

SEQ ID NO: 12 - DNA, 22 bp, Artificial Sequence
  Description of Artificial Sequence Synthetic primer
acttcaacaagcccacaggcta

SEQ ID NO: 13 - DNA, 22 bp, Artificial Sequence
  Description of Artificial Sequence Synthetic primer
tccttctgagtcttgggcatgt

SEQ ID NO: 14 - DNA, 20 bp, Artificial Sequence
  Description of Artificial Sequence Synthetic primer
atgggacgccaaggtcgtcg

SEQ ID NO: 15 - DNA, 24 bp, Artificial Sequence
  Description of Artificial Sequence Synthetic primer
tcgggtcaatgcacacttgtctgt

SEQ ID NO: 16 - DNA, 20 bp, Artificial Sequence
  Description of Artificial Sequence Synthetic primer
gagcagaccggctgatgact

SEQ ID NO: 17 - DNA, 22 bp, Artificial Sequence
  Description of Artificial Sequence Synthetic primer
agagatggccaaggcatttaac

SEQ ID NO: 18 - DNA, 15 bp, Artificial Sequence
  Description of Artificial Sequence Synthetic probe
agaggtgcaaagaca

SEQ ID NO: 19 - DNA, 18 bp, Artificial Sequence
  Description of Artificial Sequence Synthetic primer
cgcagaccgcctgatgac

SEQ ID NO: 20 - DNA, 20 bp, Artificial Sequence
  Description of Artificial Sequence Synthetic primer
gcgatggctagagcgttcac

SEQ ID NO: 21 - DNA, 14 bp, Artificial Sequence
  Description of Artificial Sequence Synthetic probe
agcgttgcaaagag

SEQ ID NO: 22 - DNA, 20 bp, Artificial Sequence
  Description of Artificial Sequence Synthetic primer
caaaccggctgagagctttc

SEQ ID NO: 23 - DNA, 16 bp, Artificial Sequence
  Description of Artificial Sequence Synthetic primer
agccgacgcggaggat

SEQ ID NO: 24 - DNA, 16 bp, Artificial Sequence
  Description of Artificial Sequence Synthetic probe
aggtcatcgagactca

SEQ ID NO: 25 - DNA, 20 bp, Artificial Sequence
  Description of Artificial Sequence Synthetic primer
cgtcactacctggcctcaca

SEQ ID NO: 26 - DNA, 19 bp, Artificial Sequence
  Description of Artificial Sequence Synthetic primer
ccccctggctgaagcatat

SEQ ID NO: 27 - DNA, 23 bp, Artificial Sequence
  Description of Artificial Sequence Synthetic probe
ccagcactacatgctccgggcaa

SEQ ID NO: 28 - DNA, 21 bp, Artificial Sequence
  Description of Artificial Sequence Synthetic primer
caaaaacgaaagcgcaagaaa

SEQ ID NO: 29 - DNA, 20 bp, Artificial Sequence
  Description of Artificial Sequence Synthetic primer
cgctctgaacaaggctcaca

SEQ ID NO: 30 - DNA, 25 bp, Artificial Sequence
  Description of Artificial Sequence Synthetic probe
cccggtttaaatcctggagcgttca

SEQ ID NO: 31 - DNA, 23 bp, Artificial Sequence
  Description of Artificial Sequence Synthetic primer
cagatacaacagaatgcggttca

SEQ ID NO: 32 - DNA, 23 bp, Artificial Sequence
  Description of Artificial Sequence Synthetic primer
tgagacaagaggctggttcctat

SEQ ID NO: 33 - DNA, 22 bp, Artificial Sequence
  Description of Artificial Sequence Synthetic probe
aaccacacggccaccatgctgg

SEQ ID NO: 34 - DNA, 26 bp, Artificial Sequence
  Description of Artificial Sequence Synthetic primer
ctacaggattcaccttacaggactca

SEQ ID NO: 35 - DNA, 20 bp, Artificial Sequence
  Description of Artificial Sequence Synthetic primer
cttcctggttggctgatgct

SEQ ID NO: 36 - DNA, 20 bp, Artificial Sequence
  Description of Artificial Sequence Synthetic probe
tgattttgcccgccgtgcct

SEQ ID NO: 37 - DNA, 22 bp, Artificial Sequence
  Description of Artificial Sequence Synthetic primer
cctacaaagtcagctcgttcca

SEQ ID NO: 38 - DNA, 22 bp, Artificial Sequence
  Description of Artificial Sequence Synthetic primer
tccttctgagtcttgggcatgt

SEQ ID NO: 39 - DNA, 18 bp, Artificial Sequence
  Description of Artificial Sequence Synthetic probe
cgggcccagcgccacact

SEQ ID NO: 40 - DNA, 22 bp, Artificial Sequence
  Description of Artificial Sequence Synthetic primer
atcagttacggtaagccagtca

SEQ ID NO: 41 - DNA, 19 bp, Artificial Sequence
  Description of Artificial Sequence Synthetic primer
tggcgacatggctctcaaa

SEQ ID NO: 42 - DNA, 26 bp, Artificial Sequence
  Description of Artificial Sequence Synthetic probe
ctgagctacagatgcccctgccgatt

SEQ ID NO: 43 - DNA, 4878 bp, Artificial Sequence
  Description of Artificial Sequence Human Shh plasmid
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgctctagcccggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgagggcggatcccccgggctgcaggaattcgatatcaagcttatcgataccgtcgacctcgagggggggcccggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

(19) United States
(12) Patent Application Publication
(10) Pub. No.: US 2007/0173471 A1
(43) Pub. Date: Jul. 26, 2007

(54) Morphogen compositions and methods of use thereof to treat heart disorders

(76) Inventor: Douglas W. Losordo, Boston, MA (US)

Correspondence Address: EDWARDS ANGELL PALMER & DODGE LLP, P.O. BOX 55874, BOSTON, MA, 02205, US

(21) Appl. No.: 11/339,808

(22) Filed: Jan. 24, 2006

(51) Int. Cl. A61K 48/00; A61K 38/17; A61F 2/02

(52) U.S. Cl. 514/44; 424/423; 514/12

(57) ABSTRACT

Disclosed are compositions and methods for preventing, treating, or reducing symptoms associated with a myocardial or related disorder such as an infarction. In one embodiment, the method includes administering a therapeutically effective amount of a nucleic acid encoding at least one morphogen; or an effective fragment thereof. Preferred morphogens include the human Sonic Hedghog (Shh), human Desert Hedgehog (Dhh), and human Indian Hedgehog (Ihh) proteins. The methods can be used alone or in combination with other methods involving administration of an angiogenic protein, hematopoietic protein, or endothelial precursor cells (EPCs).

### CROSS-REFERENCE TO RELATED APPLICATION

The present application claims priority to PCT/US2004/022328, which claims benefit from U.S. Provisional Patent Application No. 60/490,064 filed on Jul. 24, 2003, the disclosures of each of which are hereby incorporated by reference.

### STATEMENT AS TO FEDERALLY SUPPORTED RESEARCH

The present invention was made with United States government support under National Institutes of Health (NIH) grant numbers HL63414, HL63695, and HL57516. Accordingly, the United States government may have certain rights to the invention.

### FIELD OF THE INVENTION

The invention generally relates to compositions and methods for preventing or treating a heart disorder or related ailment. In one aspect, the method includes administering to a mammal a therapeutically effective amount of a nucleic acid encoding at least one morphogen; or an effective fragment thereof to prevent or treat the disorder. The invention can be used alone or in combination with recognized therapies such as further administering at least one of an angiogenic or hematopoietic protein, endothelial cells (ECs) and endothelial precursor cells (EPCs).

### BACKGROUND

There is recognition that cardiovascular disease is a major cause of mortality in the United States and claims approximately 1 million lives each year. The majority of cardiovascular deaths are due to coronary artery disease (CAD). In addition to these dire mortality statistics, CAD results in new or recurrent myocardial ischemia (MI) in approximately 1.1 million individuals each year in the United States. Of these individuals approximately 40% will go on to develop significant heart failure. A variety of strategies has been used in attempts to address the clinical consequences of loss of cardiac muscle mass, including medical treatment, revascularization to limit residual ischemia and, more recently, cardiac resynchronization therapy. In spite of these measures there were nearly one million hospitalizations for congestive heart failure in 2000, underscoring the fact that few if any therapies can fully compensate for the loss of myocardial integrity.

Intramuscular transfection of genes encoding single angiogenic cytokines may constitute a new treatment strategy for such patients (Isner and Losordo 1999). This strategy is designed to promote the development of supplemental collateral blood vessels that will constitute endogenous bypass conduits around occluded native arteries to prevent cardiac ischemia, and potentially restore myocardial function. There have been reports that VEGF gene transfer may have a favorable effect on the recovery of myocardial ischemia in humans (Losordo et al. 2002) and there are indications that this may be associated with an increase in myocardial performance (Vale, 2001a,b).

Autologous cell therapy using endothelial progenitor cells (EPCs) and bone marrow cells has also been shown to have potential for reducing myocardial ischemia and improving cardiac performance post-MI (Perin et al. 2003; Assmus et al. 2002; Kawamoto et al. 2003). Circulating EPCs derived from bone marrow mobilize endogenously in response to tissue ischemia and/or exogenously by cytokine stimulation (Takahashi et al. 1999). Locally administered stromal derived factor-1α (SDF-1α), one of the trafficking chemokines for hematopoietic stem cells, has also been shown to augment the recruitment of EPC in animal models of hindlimb ischemia (Yamaguchi et al. 2003).

It is increasingly appreciated that a potential exists to utilize signaling pathways active during embryonic development to effect therapeutic repair mechanisms in adult tissues. An exemplary pathway of this type is activated by genes of the Hedgehog (Hh) gene family, originally reported in Drosophila as a critical regulator of cell-fate determination during embryogenesis (Nusslein-Volhard C, 1980). Hh genes act as morphogens in a wide variety of tissues during embryonic development (Wang et al 1995; Roelink et al. 1994; Goodrich and Scott, 1998) primarily via actions upon mesoderm in epithelial/mesenchymal interactions that are crucial to limb, lung, gut, hair follicle and bone formation (Johnson 1997; Pepicelli et al. 1998; Ramalho-Santos et al. 2000; St-Jacques et al. 1998, 1999).

Three members of the mammalian Hh family have been reported. They are Sonic hedgehog (SHh), Desert hedgehog (DHh) and Indian hedgehog (IHh). Among these three highly conserved mammalian Hh genes, Sonic hedgehog (SHh) is the most widely expressed during development and the best studied (Zardoya et al. 1996; Bitgood et al. 1995). Hh signaling occurs through the interaction of Hh ligand with its receptor, patched-1 (Ptc-1). Once Hh binds the Ptc-1 receptor, subsequent activation of Smoothened (Smo) initiates signaling events that lead to the regulation of transcriptional factors belonging to the Gli family, which modify the expression of downstream target genes (Kogerman et al. 1999; Sisson et al. 1997).

It has been reported that human SHh protein has an indirect but robust angiogenic effect in a mouse hindlimb ischemia model via upregulation of multiple angiogenic cytokines from interstitial mesenchymal cells such as fibroblasts (Pola et al. 2001). These observations indicate that the Hh pathway can trigger a cascade of downstream trophic factors with capacity to enhance the recovery response via upregulation of multiple targets and may therefore offer advantages over approaches employing a single factor to enhance recovery from ischemic disorders.

It would be desirable to use at least one of the Sonic hedgehog (SHh), Desert hedgehog (DHh) and Indian hedgehog (IHh) proteins (or effective fragments thereof) to prevent or treat myocardial ischemia. It would be particularly useful to administer a nucleic acid encoding the human SHh plasmid to prevent or treat acute myocardial ischemia in a human patient.

### SUMMARY OF THE INVENTION

The invention generally relates to a method for preventing or treating a heart disorder in a mammal or related medical indication. In one aspect, practice of the invention involves administering to the mammal a therapeutically effective amount of a nucleic acid encoding at least one morphogenic protein; or an effective fragment thereof. The mammal can be one that has, is suspected of having, or is at risk of developing at least one of these disorders.

We have discovered that heart and related disorders can be prevented or treated by administering a therapeutically effective amount of at least one nucleic acid that encodes at least one morphogen; or an effective fragment thereof. The invention thus provides a new strategy for preventing, treating, or reducing the severity of particular disorders, especially myocardial disorders and related ailments. The invention is flexible and can be used alone or in combination with other therapies as needed. As described below, such therapies include, but are not limited to, direct administration to the mammal of a solution that includes the nucleic acid, either alone or together with administration of at least one of a morphogenic, angiogenic, and hematpoietic protein; or an effective fragment thereof. The invention further provides for administration of at least one of endothelial cells (ECs) and endothelial precursor cells (EPCs) which is believed to assist practice of the invention in certain settings. Without wishing to be bound to theory, it has been found that administration of a morphogen according to the invention facilitates a downstream cascade of one or more desirable trophic factors which can help reduce the severity of or in some cases eliminate the disorders.

Accordingly, and in one aspect, the invention provides a method for preventing, treating or reducing the severity of a myocardial disorder in a mammal. In one embodiment, the method includes administering a therapeutically effective amount of a nucleic acid encoding at least one morphogen; or an effective fragment thereof. Typical methods include selecting a mammal having the disorder and administering the nucleic acid directly to or near a heart blood vessel in need of treatment. A preferred mammal is a primate, rodent or rabbit including a human patient.

In another aspect, the invention relates to a method for inducing new blood vessel growth in myocardial tissue of a mammal in need of such treatment. In one embodiment, the method includes administering a therapeutically effective amount of a nucleic acid including encoding at least one morphogen; or an effective fragment thereof. Typically preferred invention methods include selecting a patient having the disorder and administering the nucleic acid directly to or near a heart blood vessel in need of treatment.

Further provided by the present invention is a pharmaceutical product for preventing or treating a myocardial disorder in a mammal. In one embodiment, the product includes at least one nucleic acid encoding a morphogen or effective fragment thereof, formulated to be physiologically acceptable to a mammal. The nucleic acid in the pharmaceutical product can, in one embodiment, be in the form of a plasmid or viral vector suitable for gene therapy. For some invention embodiments, the morphogen-encoding nucleotide sequence can encode a fragment of the morphogenic protein that is secreted from a cell. In preferred embodiments of the product, the morphogen or effective fragment thereof is selected from human sonic hedgehog, human desert hedgehog and human indian hedgehog. Optionally, the product further includes means for administering the product to the mammal such as a stent, catheter, or implementation for practicing angioplasty. Typical products of the invention are sterile and can further include at least one angiogenic or hematopoietic protein; or a nucleic acid encoding these proteins.

Also provided by the present invention is a kit for the introduction of at least one morphogen into a mammal. In one embodiment, the kit includes at least one nucleic acid encoding a morphogen or effective fragment thereof and optionally at least one angiogenic or hematopoietic protein or nucleic acid encoding the same. Further kits according to the invention include a pharmacologically acceptable carrier solution, and means for delivering at least the morphogen to the mammal and directions for using the kit. Examples of such means include a stent, catheter; syringe or related device such as those employed in angioplasty.

As discussed, the invention features a method for preventing or treating a myocardial or related disorder that includes administering an effective morphogen fragment. Particular fragments are discussed herein and include the N-terminal portion of the human hedgehog (Shh) protein, human desert hedgehog (Dhh) or human Indian hedgehog (Ihh) protein. As shown below, the method can be used to enhance cardiac neovascularization, reduce cardiac fibrosis, prevent cardiac apoptosis, and increase contribution of bone marrow derived endothelial progenitor cells (EPCs). These and other features of the invention provide several benefits including providing a new therapeutic approach to treating myocardial ischemia including acute and chronic forms of the disease. As will be appreciated, this aspect of the invention is flexible and can be used alone or in combination with other methods for preventing, treating, reducing the symptoms of, or delaying onset of a myocardial infarction.

Accordingly, the invention provides a method for preventing or treating a myocardial or related disorder that includes administering to a subject a therapeutically effective amount of a nucleic acid encoding an N-terminal portion of at least one of human hedgehog (Shh) protein, human desert hedgehog (Dhh) protein and human Indian Hedgehog (Ihh) protein. Preferably, the method involves administration of one or two of such nucleic acids.

In another aspect, the invention provides a method for inducing new blood vessel growth in myocardial tissue of a mammal in need of such treatment. In one embodiment, the method includes administering a therapeutically effective amount of a nucleic acid encoding an N-terminal portion of at least one of human hedgehog (Shh) protein, human desert hedgehog (Dhh) protein and human Indian Hedgehog (Ihh) protein. Preferably, the method involves administration of one or two of such nucleic acids.

Further provided by the invention is method for increasing cardiomyocyte proliferation. In one embodiment, the method involves contacting the cells with a morphogen or an effective fragment thereof. Practice of the method can be in vitro (eg., in a cell or tissue culture) or in vivo. In a typical embodiment, the method includes the steps of contacting the cardiomyocytes with an effective amount of at least one of an N-terminal portion of at least one of human hedgehog (Shh) protein, human desert hedgehog (Dhh) protein and human Indian Hedgehog (Ihh) protein. Preferably, the method involves administration of one or two of such nucleic acids.

The invention also provides a method for increasing production of endothelial precursor cells (EPCs). In one embodiment, the method includes administering a therapeutically effective amount of of at least one of an N-terminal portion of at least one of human hedgehog (Shh) protein, human desert hedgehog (Dhh) protein and human Indian Hedgehog (Ihh) protein to a subject; and increasing production of the EPCs. Preferably, the method involves administration of one or two of such nucleic acids to increase production of the EPCs.

Further provided is a method for increasing production of a cytokine either in vitro (eg., in a cell or tissue culture) or in vivo. In one embodiment, the method includes administering a therapeutically effective amount of at least one of an N-terminal portion of at least one of human hedgehog (Shh) protein, human desert hedgehog (Dhh) protein and human Indian Hedgehog (Ih) protein to a subject; and increasing production of the cytokine. Preferably, the method involves administration of one or two of such nucleic acids.

Other uses and advantages of the invention are discussed below.

### BRIEF DESCRIPTION OF THE DRAWINGS

FIGS. 1A-C. (A) is a graph showing Shh mRNA upregulation after myocardial ischemia; (B) is two photographs of hearts showing upregulation of Ptc-1 after myocardial ischemia, and (C) is a Western blot of Ptc-1 protein expression in ischemic and non-ischemic myocardium.

FIGS. 2A-L. (A) is an immunoblot showing expression of Shh protein by transfected COS cells; (B) is a gel showing Gli mRNA after plasmid transfection; (C) is two photographs showing heart whole mount X-gal staining of Ptc1 after phShh or control plasmid injection; (D) is Western blot showing Ptc1 protein expression seven days after plasmid injection; (E) and (F) are fluorescence micrographs showing Pct1 expression by fibroblasts and cardiomyocytes; (G-L) are graphs showing quantitative RT-PCR analysis of transcripts in cultured fibroblasts treated with Shh conditioned medium.

FIGS. 3A-I. (A) and (B) are two photographs of rat heart sections and (C) is a graph showing assessment of myocardial infarction in rats treated with phShh or control plasmid; (D-F) are two micrographs (D,E) of ischemic heart sections immunostained for isolectin B4 and a graph (F) showing quantitation of capillary density in these hearts; (G-I) are two micrographs immunostained for smooth muscle action (G, H) and a graph (I) showing quantitation of smooth muscle acting area.

FIGS. 4A-F. (A-F) are six graphs showing assessment of heart function in rats at intervals after myocardial ischemia and injection with phShh or control plasmids. (A-C) show echocardiographic parameters: (A), fractal shortening (FS), (B), left ventricular diastolic dimension(LVDd) and (C), wall motion score change; (D-F) show hemodynamic measures: (D) developed pressure, (E) left ventricular positive and negative pressure and (F) instantaneous pressure.

FIGS. 5A-F. (A-C) are representative recordings (A,B) of NOGA electrochemical mapping before(pre transfer) and 4 weeks after (post transfer) plasmid injection and a graph (C) showing phShh mediated reduction in ischemic area; (D-F) are five photographs (D,E) showing left coronary angiography pre and post transfer and a graph (F) showing Rentrop score of collateral development.

FIGS. 6A-F. (A-C) are four photographs (A) of X-gal stained heart wholemounts, and two photomicrogaphs (B) of heart sections showing X-gal staining of endothelial progenitor cells (EPC), and a graph (C) showing quantitation of X-gal positive cells representing bone-marrow derived EPCs; (D-F) are three fluorescence micrographs showing bone marrow derived EPCs incorporated into new blood vessels following treatment with phShh.

FIGS. 7A-D. (A) and (C) are six fluorescence micrographs (A) showing activated caspase3 immunostaining in cardiomyocytes and a graph (C) showing quantitation of active caspase3 area in hearts injected with phShh and control plasmid; (B) and (D) are three fluorescence micrographs (B) and a graph (D) showing quantitation of apoptosis by TUNNEL positivity in cultured cardiomyocytes treated with Shh protein.

FIGS. 8A-H. (A) and (B) are four fluorescence micrographs (A) showing immunostaining of proliferating cardiomyocytes and a graph (B) showing quantitation of BrdU-positive cardiomyocytes in hearts injected with control and phShh plasmids; (C-E) are eight micrographs (C,D) showing immunostaining of Pct1, α-actinin, DAPI and BrdU and a graph (E) showing quantitation of BrdU-positive cardiomyocytes in vitro following treatment with Shh; (F-H) are two micrographs (F) and two graphs (G, H) showing increased proliferative activity in cultured cardiomyoblasts

FIGS. 9A-C. (A) is four fluorescence micrographs showing immunostaining of Ptc1 and α-actinin in cultured cardiomyoblasts; (B) is a Western blot showing Ptc1 protein expression in the nucleus and cytoplasm of cardiomyoblasts; and (C) is a gel showing Gli-1 mRNA expression by RT-PCR in these cells.

FIGS. 10A-C is a drawing showing the sequence of the human Shh plasmid. Sequence encoding the N-terminal portion of the protein is underlined.

### DETAILED DESCRIPTION OF THE INVENTION

As discussed, the invention can be used to prevent or treat a myocardial disorder in a mammal such as a human patient or a related ailment. Typical invention methods include administering a therapeutically effective amount of at least one nucleic acid to a patient in need of such treatment which nucleic acid encodes at least one morphogen; or an effective fragment of that morphogen. Sometimes but not exclusively, the method will involve administering one nucleic acid to the patient which will preferably encode one morphogen or an effective fragment thereof.

In broad terms, morphogens are recognized amino acid sequences whose concentration is read by cells as positional information to a pre-determined landmark or beacon in certain cells. Such sequences help control pattern formation in a large field of adjacent tissue. See generally Alberts, B. et al. (1989) in the Molecular Biology of the Cell, 2^{nd} Ed. Garland Publishing, New York, N.Y. (discussing morphogen function). As used herein, “morphogen” or related phrase such as “morphogenic protein” means one of the following mammalian: Sonic hedgehog (SHh), Desert hedgehog (DHh) and Indian hedgehog (IHh); or an effective fragment thereof Preferred morphogens are endogenous to a primate and especially a human. A more preferred morphogen is human SHh.

There are reports that most morphogens and especially SHh are protein signaling molecules important for vertebrate patterning. In particular, SHh has been shown to be involved in the morphogenesis of many vertebrate organ systems. The sequence of many mammalian morphogens has been disclosed. For example, the human SHh sequence (nucleic acid and protein) has been reported eg., by Margio, V. (1995) as a submission to the National Center For Biotechnology Information (NCBI) Genetic Sequence Data Bank (Genbank) at the National (U.S) Library of Medicine, 38A, 8N05, Rockville Pike, Bethesda, Md. 20894. See eg., accession number L38518 (version L38518.1; GI: 663156) which reports a 1576 basepair human SHh nucleic acid sequence and corresponding encoded protein. It will be appreciated that any nucleic acid or protein sequence information not specifically disclosed herein can be readily accessed at Genbank, EMBL and/or SWISS-PROT. Using this information, a DNA or RNA segment encoding the desired may be chemically synthesized or, alternatively, such a DNA or RNA segment may be obtained using routine procedures in the art, e.g, PCR amplification. See also Pola, R. et al. (2003) Circulation 108: 479-485 (Jul. 14, 2003 edition; PUBMED ID NO. 12860919) in which an especially preferred SHh vector construct is disclosed.

By the phrase “effective fragment” is meant a nucleic acid or amino acid sequence that has at least 80% of the biological function of the corresponding full-length sequence, preferably at least about 90% and more preferably at least about 95% of that function. By an “effective fragment” of a morphogen or related phrase is meant a portion of a morphogen protein or nucleic acid encoding same that has at least 80% of the activity of the corresponding full-length protein or nucleic acid as determined by what is referred to herein as a “standard heart LV function assay”. A preferred version of that assay includes performing at least one of and preferably all of the following steps:
(a) directly injecting into a rodent subjected to LAD ligation a solution that includes therein a nucleic acid (about 1 μg/ml to about 5 mg/ml) encoding the desired portion of the morphogen,
(b) allowing the rodent to recover from the LAD ligation for at least about few hours, preferably about a few days or more, more preferably about a few weeks up to about one to two months; and
(c) evaluating function of the rodent heart e.g., by performing transthoracic echocardiography or related procedure.

A preferred embodiment of the assay is discussed below in the Examples section.

Also contemplated for use with the present invention are morphogen “derivatives” which include a morphogen analogue having substantial identity to the corresponding full-length morphogen. Preferred derivatives are at least about 90% identical to the full-length morphogen as determined eg., by inspection or with the aid of a suitable computer program such as BLAST, FASTA or related programs, preferably at least about 95% identical. Suitable analogues include protein sequences having one or more conservative amino acid substitutions with respect to the corresponding full-length morphogen (eg., allelic variants). By “conservative” amino acid substitution is replacement of one amino acid residue for another having similar chemical properties (eg., replacing tyrosine with phenylalanine).

In embodiments in which prevention or treatment of a myocardial disorder is desired, the invention can further include selecting a patient having the disorder and administering the nucleic directly to or near a heart blood vessel in need of treatment. Typically such methods include the step of expressing the morphogen or fragment directly to or near the blood vessel in the patient to prevent or treat the myocardial disorder.

Methods for administering a nucleic acid to a mammal and particularly direct injection to or near the heart and associated tissue have been disclosed. See e.g., U.S. Pat. Nos. 5,830,879; 6,258,787; 6,121,246; RE37,933, 5,851,521 and 5,106,386; the disclosures of which are incorporated herein by reference.

In one approach, and to simplify the manipulation and handling of the nucleic acid encoding the morphogen or effective fragment, the nucleic acid is preferably inserted into a cassette where it is operably linked to a promoter. The promoter must be capable of driving expression of the mitogen in the desired target host cell. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the beta.-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/122618.

If desired, the DNA may also be used with a microdelivery vehicle such as cationic liposomes and adenoviral vectors. For a review of the procedures for liposome preparation, targeting and delivery of contents, see Mannino and Gould-Fogerite, Bio Techniques, 6:682 (1988). See also, Feigner and Holm, Bethesda Res. Lab. Focus, 11 (2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

In embodiments in which prevention or treatment of a myocardial disorder is desired, a blood vessel or associated tissue is contacted with the DNA encoding the morphogen or effective fragment by conventional means such as, but not limited to, double-balloon catheters, porous-balloon catheters and hydrophilic coated balloon catheters. See, Jorgensen, et al., Lancet 1:1106-1108, (1989); Wolinsky, et al., J. Am. Coll. Cardiol., 15:475-485 (1990); March, et al., Cardio Intervention, 2:11-26 (1992)); WO93/00051 and WO93/00052; U.S. Pat. No. 5,304,121. See also Dichek, Textbook of interventional Cardiology, Vol. 2, 61:989-1005.

Thus in one embodiment, the present invention method further includes administering the nucleic acid with a stent, catheter, implementation for performing balloon angioplasty; or related device. Preferred myocardial tissue for use with the invention is known, suspected to be, or at risk of being impacted by one or more of ischemia, infarction or dysfunction.

If desired, the methods disclosed herein can be used alone or in combination with other recognized therapies that promote blood vessel growth. See eg., EP1061800 and WO99/45775 (disclosing eg., compositions and method for modulating vascularization). In one embodiment, such methods include administering to the heart blood vessel of a human patient in need of such treatment at least one nucleic acid encoding at least one of an angiogenic or hemaotpoietic protein; or effective fragment thereof.

Preferred angiogenic protein include but are not limited to acidic fibroblast growth factor (aFGF), basic fibroblast growth factor (bFGF), vascular endothelial growth factor (VEGF-1), epidermal growth factor (EGF), transforming growth factor α and β (TGF-α and TFG-β), platelet-derived endothelial growth factor (PD-ECGF), platelet-derived growth factor (PDGF), tumor necrosis factor α (TNF-α), hepatocyte growth factor (HGF), insulin like growth factor (IGF), erythropoietin, colony stimulating factor (CSF), macrophage-CSF (M-CSF), angiopoetin-1 (Ang1) or nitric oxidesynthase (NOS); or an effective fragment thereof.

Preferred hematopoietic proteins include granulocyte-macrophage colony-stimulating factor (GM-CSF), VEGF, Steel factor (SLF, also known as Stem cell factor (SCF)), stromal cell-derived factor (SDF-1), granulocyte-colony stimulating factor (G-CSF), HGF, Angiopoietin-1, Angiopoietin-2, M-CSF, b-FGF, and FLT-3 ligand.

Methods for testing and identifying a variety of suitable angiogenic and hematopoietic protein fragments have been reported in EP1061800 and WO99/45775, for instance.

Once transferred to or near the site of a heart blood vessel, the nucleic acid (usually DNA) is expressed by the myocardial cells for a period of time sufficient to assist new vascularization (angiogenesis). Certain vectors in accord with the invention may not normally be incorporated into the genome of the cells. In these embodiments, expression of the morphogen or effective fragment thereof of interest takes place for only a limited time. In such embodiments, the morphogen or fragment is only expressed in therapeutic levels for about two days to several weeks, for instance about 1-2 weeks up to about one to two months. Reapplication of the DNA can be utilized to provide additional periods of expression of the therapeutic morphogen.

Suitable vehicles, be it arterial balloon, catheter, flexible rod or other shaped vehicle, can be furnished with means to assist in accurate placement within the intended body cavity including the thoracic cavity. For example, it can be furnished with a radioactive element, or made radio-opaque, furnished with means permitting easy location using ultrasound, etc.

As discussed, the invention is flexible and it can be used to treat other blood vessel injuries. For example, primary angioplasty is becoming widely used for the treatment of acute myocardial infarction. Acceleration of reendothelialization using the method of the present invention can help maintain existing vasculature.

In another invention embodiment, the methods described herein can further include administering to the mammal an effective amount of at least one morphogenic protein or an effective fragment thereof.

The term “effective amount” as used herein and especially a “therapeutically effective amount” means a sufficient amount of nucleic acid delivered to the desired cells or tissue to produce an adequate level of the morphogen or effective fragment, i.e., levels capable of inducing endothelial cell growth and more generally, blood vessel growth. Thus, the important aspect is the level of morphogen expressed. Accordingly, one can use multiple transcripts or one can have the nucleic acid coding sequence under the control of a promoter that will result in high levels of expression. In an alternative embodiment, nucleic acid coding sequence would be under the control of a factor that results in extremely high levels of expression, e.g., tat and the corresponding tar element. For instance, administration of between about 1 pg/g organ weight to 1 mg/organ weight will suffice for many invention embodiments.

As also discussed, the invention features a method for inducing new blood vessel growth in myocardial tissue of a mammal in need of such treatment. In one embodiment, the method includes administering a therapeutically effective amount of a nucleic acid encoding at least one morphogen; or an effective fragment thereof. Typically, such a method further includes selecting a patient having the disorder and administering the nucleic to or near a heart blood vessel in need of treatment. Also typically, the method includes expressing the morphogen or fragment to or near the blood vessel in the patient to prevent or treat the myocardial disorder. Any suitable method of administering the nucleic acid, such as those methods already described, can be used.

According to the invention method, the myocardial tissue can be impacted by one of ischemia, infarction or dysfunction. It may further include administering to the heart blood vessel of the patient at least one nucleic acid encoding at least one of an angiogenic or hemaotpoietic protein; or effective fragment thereof as described previously. Alternatively, or in addition, the method can further include the step of administering to the mammal an effective amount of at least one morphogenic protein or an effective fragment thereof.

There are reports that new blood vessel growth can be facilitated by administering to a mammal a preparation that includes endothelial cells (ECs), endothelial progentitor cells (EPCs) or both. Thus the present invention further provides for a method in which administration of nucleic acid encoding the morphogen or effective fragment additionally includes administering a therapeutically effective amount of endothelial cells (ECs) or endothelial cell precursors (EPCs). Preferred ECs and EPCs are characterized by having at least one of the following markers: CD34⁺, flk-1⁺, and tie-2⁺. See eg., U.S. Pat. Nos. 6,659,428; 5,980,887; EP1061800; WO 99/45775; U.S. Pat. Nos. 5,830,879; 6,258,787; 6,121,246; RE37,933, 5,851,521 and 5,106,386, (disclosing methods for preparing and using ECs and EPCs to promote blood vessel growth).

As mentioned the invention features a pharmaceutical product for preventing or treating a myocardial disorder in a mammal. In one embodiment, the product includes at least one morphogenic protein or effective fragment thereof formulated to be physiologically acceptable to a mammal. Preferably, the product further includes means for administering the product to the mammal such as those already mentioned. Optionally, the product is sterile and further includes at least one of an angiogenic or hematopoietic protein; or nucleic acid encoding the protein. If desired, the pharmaceutical product can further include endothelial cells (ECs), endothelial progenitor cells (EPCs) or both cell types.

Further provided by the invention is a kit for the introduction of at least one morphogen into a mammal. Preferred kits include at least one morphogen or effective fragment thereof and optionally at least one angiogenic or hematopoietic protein or nucleic acid encoding same. In one embodiment, the kit further includes a pharmacologically acceptable carrier solution, and means for delivering at least the morphogen to the mammal and directions for using the kit. If desired, the pharmaceutical product can further include endothelial cells (ECs), endothelial progenitor cells (EPCs) or both cell types. Preferably in such embodiments, the kit further includes means for delivering the cells to the mammal such as a stent, catheter; syringe or related device.

As discussed, the invention provides a method for preventing or treating a myocardial or related disorder. In one embodiment, the includes administering a therapeutically effective amount of a nucleic acid encoding an N-terminal portion of at least one of human hedgehog (Shh) protein, human desert hedgehog (Dhh) protein and human Indian Hedgehog (Ihh) protein.

It has been reported that Human Shh is synthesized as a 45 kDa precursor protein that is cleaved autocatalytically to yield two “effective fragments” ie., (1) a 20 kDa N-terminal fragment that is responsible for all known hedgehog signaling activity ; and (2) a 25 kDa C-terminal fragment that contains the autoprocessing activity. The N-terminal fragment is reported to consist of amino acid residues 24-197 of the full-length precursor sequence. The N-terminal fragment is thought to remain membrane-associated through the addition of a cholesterol at its C-terminus. The addition of the cholesterol is catalyzed by the C-terminal domain during the processing step. See eg., U.S. Pat. Nos. 6,132,728; 6,281,332; 6,444,793; and 6,288,048 as well as WO 95/18856; and WO 96/17924.

The complete amino acid sequences of the human Dhh and human Ihh proteins have been reported by Genbank as accession nos. BC033507 and BC034757, respectively. Also disclosed are the corresponding cDNA sequences. Significant relationship between the N-terminal fragments of the human Shh, human Dhh, and human Ihh proteins has been reported. See U.S. Pat. No. 6,444,793, for instance.

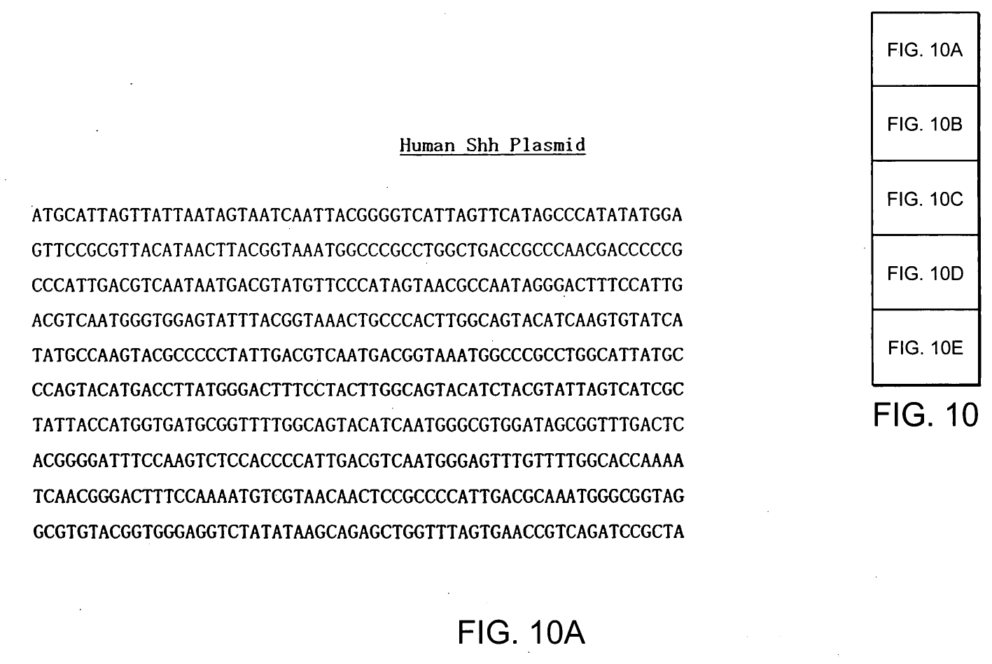

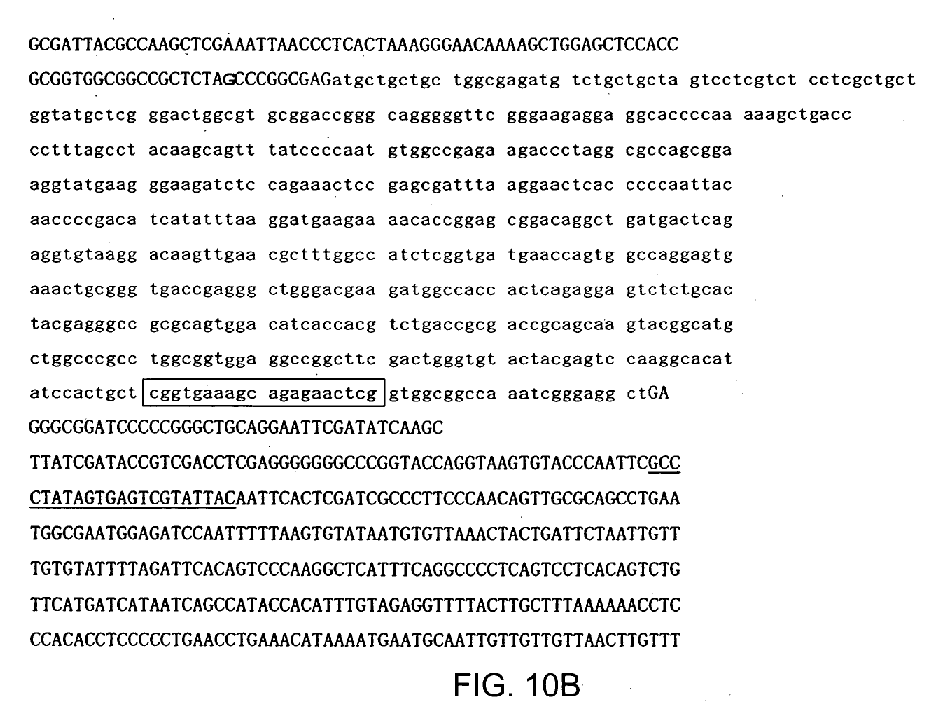

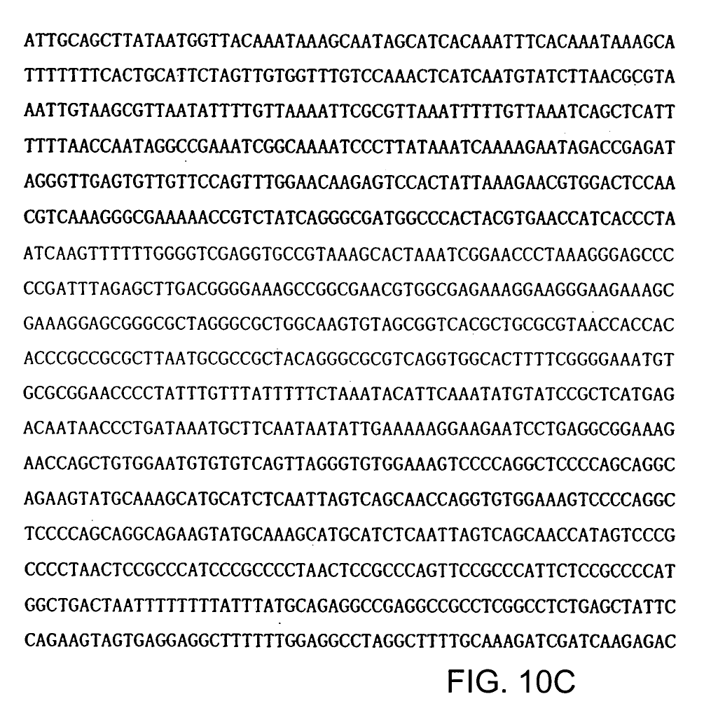

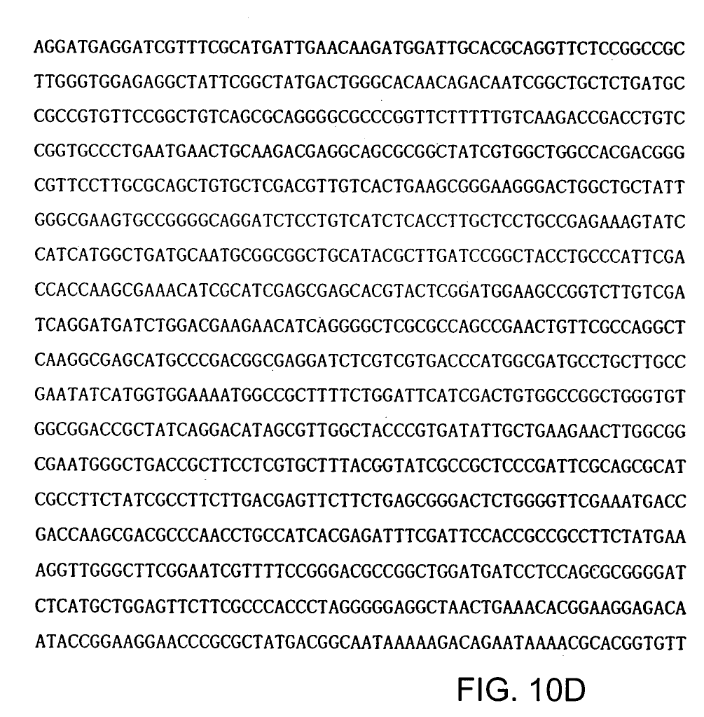

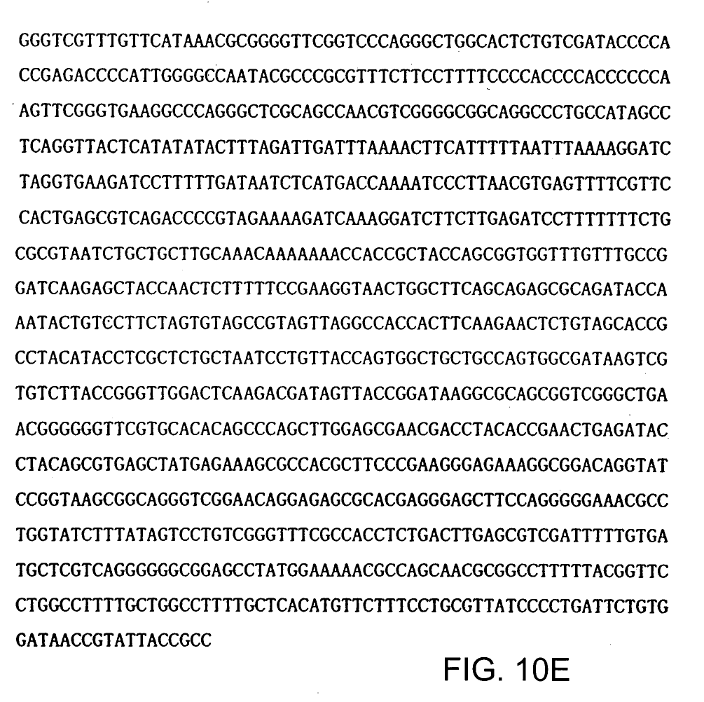

By the term “N-terminal portion” of the human Shh protein is meant between from about 400 bp to about 700 bp amino terminal domain coding sequence of the human Shh gene, preferably about the 600 bp amino terminal domain coding sequence. See Table I, below and FIG. 10A-C, for instance. By the phrase “N-terminal portion” of the human Dhh and human Ihh proteins is meant less than about an 800 bp amino terminal domain coding sequence, preferably less than about 700 bp, more preferably between from about 300 bp to about 600 bp of the amino terminal domain coding sequence for the human Dhh and human Ihh proteins. Fragment lengths are generally measured from the N-terminus of the mature protein (before autocatalysis). Various other effective N-terminal fragments that encompass the N-terminal moiety are considered within the presently claimed invention. Publications disclosing these sequences, as well as certain of their chemical and physical properties, include WO 95/23223; WO 95/18856; and WO 96/17924; and U.S. Pat. No. 6,444,793. Preferred of such encoded N-terminal portions of the human Shh, Dhh, and Ihh proteins are those that exhibit at least 80% of the activity of the corresponding full-length protein as determined by the standard heart LV function assay described above.

Further effective portions of the human Shh, Dhh, and Ihh proteins are contemplated and include those are at least 80% identical to the N-terminal portion of the proteins specified above, preferably at least about 90% identical or more such as 95% identical. Specifically included are allelic variants of such proteins as well as conservative amino substitutions of such proteins (eg., alanine for serine, glycine for alanine, etc.)

See the following references for disclosure relating to still further morphogens and effective fragments thereof suitable for use with the invention. Lee et al. (1994) Science 266:1528-1537; Porter et al. (1995) Nature 374:363-366); Bumcrot, D. A., et al. (1995) Mol. Cell. Biol. 15:2294-2303; Ekker, S. C. et al. (1995) Curr. Biol. 5:944-955; and Lai, C. J. et al. (1995) Development 121:2349-2360).

Table I, below, shows the amino acid sequence of an especially preferred N-terminal amino portion of the human Shh gene sequence (A). See also FIG. 10A-C. Illustrative N-terminal portions of the human Dhh (B) and human Ihh gene (C) sequences are also shown.

Sequence for (B) and (C) was taken from Genbank accession nos. BC033507 and BC034757, respectively. In the case of the human Ihh protein sequence, the first amino amino acid in the mature protein sequence is reported by Genbank to be glutamic acid.

TABLE I

Illustrative N-terminal portions of human
Shh, Dhh, and Ihh genes

A. atgctgctgc tggcgagatg tctgctgcta gtcctcgtct
cctcgctgct ggtatgctcg ggactggcgt gcggaccggg
cagggggttc gggaagagga ggcaccccaa aaagctgacc
cctttagcct acaagcagtt tatccccaat gtggccgaga
agaccctagg cgccagcgga aggtatgaag ggaagatctc
cagaaactcc gagcgattta aggaactcac ccccaattac
aaccccgaca tcatatttaa ggatgaagaa aacaccggag
cggacaggct gatgactcag aggtgtaagg acaagttgaa
cgctttggcc atctcggtga tgaaccagtg gccaggagtg
aaactgcggg tgaccgaggg ctgggacgaa gatggccacc
actcagagga gtctctgcac tacgagggcc gcgcagtgga
catcaccacg tctgaccgcg accgcagcaa gtacggcatg
ctggcccgcc tggcggtgga ggccggcttc gactgggtgt
actacgagtc caaggcacat atccactgct cggtgaaagc
agagaactcg gtggcggcca aatcgggagg ct
B. atg gctctcctga ccaatctact gcccctgtgc tgcttggcac
ttctggcgct gccagcccag agctgcgggc cgggccgggg
gccggttggc cggcgccgct atgcgcgcaa gcagctcgtg
ccgctactct acaagcaatt tgtgcccggc gtgccagagc
ggaccctggg cgccagtggg ccagcggagg ggagggtggc
aaggggctcc gagcgcttcc gggacctcgt gcccaactac
aaccccgaca tcatcttcaa ggatgaggag aacagtggag
ccgaccgcct gatgaccgag cgttgtaagg agcgggtgaa
cgctttggcc attgccgtga tgaacatgtg gcccggagtg
cgcctacgag tgactgaggg ctgggacgag gacggccacc
acgctcagga ttcactccac tacgaaggcc gtgctttgga
catcactacg tctgaccgcg accgcaacaa gtatgggttg
ctggcgcgcc tcgcagtgga agccggcttc gactgggtct
actacgagtc ccgcaaccac gtccacgtgt cggtcaaagc
tgataactca ctggcggtcc gggcgggcgg ctgctttccg
C. ggagaacaca ggcgccgacc gcctcatgac ccagcgctgc
aaggaccgcc tgaactcgct ggctatctcg gtgatgaacc
agtggcccgg tgtgaagctg cgggtgaccg agggctggga
cgaggacggc caccactcag aggagtccct gcattatgag
ggccgcgcgg tggacatcac cacatcagac cgcgaccgca
ataagtatgg actgctggcg cgcttggcag tggaggccgg
ctttgactgg gtgtattacg agtcaaaggc ccacgtgcat
tgctccgtca agtccgagca ctcggccgca gccaagacag
gcggctgctt ccctgccgga gcccaggtac gcctggagag
tggggcgcgt gtggccttgt cagccgtgag gccgggagac
cgtgtgctgg ccatggggga ggatgggagc cccaccttca
gcgatgtgct cattttcctg gaccgcgagc ctcacaggct
gagagccttc caggtcatcg agactcagga ccccccacgc
cgcctggcac tcacacccgc tcacctgctc tttacggctg
acaatcacac ggagccggca gcccgcttcc gggccacatt

Thus in one embodiment, the method further includes selecting a patient having the myocardial disorder and administering the nucleic acid encoding the Shh, Dhh, and/or Ihh protein directly to or near a heart blood vessel in need of treatment. Preferred methods generally involve the step of expressing the morphogen or fragment directly to or near the blood vessel in the patient to prevent or treat the myocardial disorder. If desired, the method can further include administering the nucleic acid with a stent, catheter, implementation for performing balloon angioplasty; or related device.

Such methods can be used to treat a variety of myocardial tissue including those tissues impacted by one of ischemia, infarction or dysfunction.

As mentioned, the foregoing methods are flexible and can be used in combination with other treatment methods. Thus in one embodiment, the invention further includes administering to the heart blood vessel of the patient at least one nucleic acid encoding at least one of an angiogenic or hemaotpoietic protein; or effective fragment thereof. If desired, the method can further include administering to the mammal an effective amount of at least one morphogenic protein or an effective fragment thereof. Alternatively, or in addition, the method can further include administering to the mammal an effective amount of at least one of an angiogenic or hematopoietic protein.

As also discussed the invention features a method for inducing new blood vessel growth in myocardial tissue of a mammal in need of such treatment. In one embodiment, the method includes administering a therapeutically effective amount of a nucleic acid encoding an N-terminal portion of at least one of human hedgehog (Shh) protein, human desert hedgehog (Dhh) protein and human Indian Hedgehog (Ihh) protein. In one further embodiment, the method includes selecting a patient having the disorder and administering the nucleic to or near a heart blood vessel in need of treatment. Preferably, the method further includes expressing the morphogen or fragment to or near the blood vessel in the patient to prevent or treat the myocardial disorder. If desired, the method can further include administering the nucleic with a stent, catheter, implementation for performing balloon angioplasty; or related device.

In another embodiment of the method, the myocardial tissue has been impacted by one of ischemia, infarction or dysfunction. If desired, the method can further include to the heart blood vessel of the patient at least one nucleic acid encoding at least one of an angiogenic or hemaotpoietic protein; or effective fragment thereof. The method may further include administering to the mammal an effective amount of at least one morphogenic protein or an effective fragment thereof. The method can further include administering to the mammal an effective amount of at least one of an angiogenic or hematopoietic protein. If desired, the method can further include administering to the patient a therapeutically effective amount of endothelial cells (ECs) or endothelial cell precursors (EPCs). Preferably, the cells are characterized by having at least one of the following markers: CD34⁺, flk-1⁺, and tie-2⁺.

Further provided by the invention is a method for increasing cardiomyocyte proliferation that includes the steps of contacting cardiomyocytes with an effective amount of at least one of an N-terminal portion of at least one of human hedgehog (Shh) protein, human desert hedgehog (Dhh) protein and human Indian Hedgehog (Ihh) protein. Suitable cells can be obtained as primary cultures or as cell lines obtained from the American Type Tissue Collection (Manassas, Va.). In one embodiment, the method is performed in vitro eg., as a way of maintaining the cells in culture. In another embodiment, the method is performed in vivo e.g., to enhance numbers of cardiomyocytes in a patient in need of such treatment.

As mentioned, the invention further provides for a method for increasing production of endothelial cell precursors (EPCs). In one embodiment, the method includes administering a therapeutically effective amount of of at least one of an N-terminal portion of at least one of human hedgehog (Shh) protein, human desert hedgehog (Dhh) protein and human Indian Hedgehog (Ihh) protein to a subject; and increasing production of the EPCs. Such EPCs can be isolated from the patient, if needed, treated ex vivo, and given back to the patient. Disclosure relating to preferred EPC isolation and ex vivo treatment methods have been disclosed in one or more of the following references: U.S. Pat. Nos. 6,676,937; 6,569,428; 6,258,787; and 5,652,225. In one embodiment, the method involves increasing neovascularization in the subject.

As discussed, the invention also features a method for increasing production of a cytokine in a subject that involves, in one embodiment, administering a therapeutically effective amount of at least one of an N-terminal portion of at least one of human hedgehog (Shh) protein, human desert hedgehog (Dhh) protein and human Indian Hedgehog (Ihh) protein to a subject; and increasing production of the cytokine. Preferred cytokines include SDF-1α, vascular endothelial growth factor (VEGF), angiopoietin-1, angiopoietin-2; IGF-1; and biologically active fragments thereof.

The invention is further illustrated by reference to the following non-limiting examples.

### EXAMPLE 1

### Preparation of Human SHh Plasmid

There have been reports that native full length SHh gene product undergoes an auto-processing reaction during its biogenesis resulting in the amino- and carboxy- terminal domain products {Roelink, 1995}. Biologic activity is contained in the amino-terminal cleavage product, however, during auto-processing, the amino-terminal domain products were cholesterol modified and this modification causes the amino-terminal protein to be tightly cell associated {Porter, 1996}, leaving the protein is tethered to the cell that made it. This phenomenon was viewed as disadvantageous for a local gene therapy approach. Thus the amino-terminal domain of human SHh was selected as coding sequence to make a SHh-plasmid using pCMV-ScriptPCR Cloning Kit (Stratagene). This plasmid of human SHh (phSHh) is a 4,878-bp plasmid that contains the 600 bp amino terminal domain of human SHh coding sequence. Expression from SHh gene is modulated by the presence of promoter sequences from cytomegalovirus to drive SHh expression. Downstream from the SHh cDNA is an SV40 polyadenylation sequence. The plasmid also contains a gene that confers neomycin/kanamycin resistance to the host cells.

### EXAMPLE 2

### Assessment of Transgene Expression In Vitro

Medium was conditioned by transfected COS cells using liposome based Transfast transfection kit (Promega) according to the manufacturers directions. After 24 hours of transfection, the supernatants were harvested and cells extracted with RIPA buffer (50 mM Tris, 150 mM sodium chloride, 1% NP40, 0.5% sodium deoxycholate, 0.1% SDD) with proteinase inhibitors (Roche Molecular Biochemicals) and centrifuged at 3,000 rpm for 20 minutes at 4° C. twice. Total protein extracts were quantified using the BioRad protein assay (BioRad, Herwles, Calif.). Total cytosolic proteins (40 μg) were electrophoresed on a 10% sodium dodecyl sulphate-polyacrylamide gel and electrophoretically transferred to an Immuno-Blot PVDF membrane (BioRad, Herwles, Calif.). Protein standards (BioRad, Herwles, Calif.) were run in each gel. The blots were blocked with 5% milk in Tris-buffered saline Tween-20 for 1 hour at room temperature. Blots were incubated overnight at 4° C. with specific primary goat antibody against SHh goat polyclonal antibody (1:1000 dilution, Sigma Chemical Co.) for 2 hours at room temperature. After stringent washing, blots were incubated for 1 hour at room temperature with 1:5000 diluted horseradish peroxidase-conjugated secondary antibodies (Santa Cruz Biotech, Santa Cruz, Calif.) (donkey anti-goat IgG) for SHh. Peroxidase activity was visualized by exposing an X-ray film to blots incubated with ECL regent (Amersham, Arlington Heights, Ill.).

The conditioned media from transfected COS cells was also incubated with cultured rat nerve fibroblasts to examine the expression of the transcriptional factor Gli-1, a downstream target gene of SHh, by RT-PCR as described below.

### EXAMPLE 3

### Animal Model of Myocardial Ischemia

A total of thirty-five 6 week old male Sprague-Dawley rats (Charles River Laboratories, Wilmington Mass.) were used for this study. We also used twenty bone marrow transplant (BMT) animal models using FVB/N-TgN[TIE2/LacZ] mice as previously described {Asahara, 1999}. Tie-2/BMT mice, which receive bone marrow from transgenic mice constitutively overexpressing beta-galactosidase regulated by the endothelial specific Tie-2 promoter. Twenty female FVB mice (4 weeks old) were used as recipients. At 4 weeks after BMT, by which time the BM of the recipient mice is typically reconstituted, BMT mice (at 8 weeks age) underwent ischemia induction by LAD ligation.

All animals were anesthetized with sodium pentobarbital (50 mg/kg IP). Animals were orally intubated with 20 GIV (rat) or 22 GIV(mice) catheter and artificially ventilated with a respirator (Harvard Apparatus). A small oblique thoracotomy was performed lateral to the midstemal line (rat) or left intercostal line (mice) in the third costal space to expose the heart. After the pericardium was opened, the proximal left anterior descending artery (LAD) branch of the left coronary artery was ligated using 6-0 (rat) or 8-0 (mice) polypropylene sutures through a dissecting microscope.

### EXAMPLE 4

### Intramyocardial Gene Delivery

Direct myocardial DNA injections were performed 10 minutes after LAD ligation. Rats received an escalating dose of the naked plasmid DNA encoding for human SHh (phSHh-30, 300 and 600 μg/0.1 ml sterile saline) subepicardially with a curved 27-gauge needle into 5 sites along the anterior and posterior left ventricular wall. Mice received one dose of phSHh (100 μg/0.02 ml sterile saline). Control animals received an equivalent volume of either sterile saline (PBS) or empty-LacZ vector (pLacZ). The area of injection corresponded to the region of the myocardium supplied by the left ascending coronary artery (LAD). The chest incision was then closed in layers with 3.0 (rat) or 6-0 (mice) suture.

### EXAMPLE 5

### Physiological Assessment of LV Function

Transthoracic echocardiography (SONOS 5500, Hewlett Packard) was performed 5, 14 and 28 days after gene delivery in the rat MI model. LV diastolic (LVDd) and systolic (LVDs) dimensions and fractional shortening (FS) were measured at the mid-papillary muscle level. Regional wall motion score was examined per published criteria. Hemodynamic measurements were obtained 4 weeks after myocardial ischemia using 2.0-French high-fidelity Millar pressure catheter (Millar Instuments, Houston) via the right carotid artery. After stabilization of LV function and heart rate, LV systolic pressure, end-diastolic pressure, developed pressure, maximal positive (+dP/dt) and negative (−dP/dt) pressure development were recorded. All procedures and analyses were performed by an experienced researcher who was blinded to treatment.

### EXAMPLE 6

### Hemodynmic Assessment

Cardiac hemodynamics in rats were assessed 28 days after injection of phSHh, pLacZ or saline. Rats were anesthetized with sodium pentobarbital (50 mg/kg, IP) and intubated. Body temperature was maintained at 37° C. A 2F high-fidelity catheter (Millar Instruments Inc., Houston, Tex.) was inserted via the right carotid artery into the left ventricle (LV) for recording of LV pressures, heart rate (HR), LV end-diastolic pressure (LVEDP), minimum and maximal rate of rise of LV pressure (dP/dtₘᵢₙ, dP/dtₘₐₓ) after stabilization period of 15 min, Calibration of the Millar catheter was verified before and after each measurement.

### EXAMPLE 7

### Histological Assessment

At necropsy, rat hearts was sliced in a bread-loaf fashion into 4 transverse sections from apex to base and fixed with 4% paraformaldehyde 28 days after gene delivery. Paraffin-embedded tissues stained by elastic-trichrome were used to measure the average ratio of fibrosis area to left ventricular (LV) area. Immunohistochemical staining was performed using antibodies prepared against the murine-specific EC marker isolectin B4 (Vector Laboratories). Capillary density was evaluated morphometrically by histological examination of 5 randomly selected fields of tissue sections recovered from segments of LV myocardium, subserved by the occluded LAD. Capillaries were recognized as tubular structures positive for isolectin B4. All morphometric studies were performed by two examiners who were blinded to treatment.

### EXAMPLE 8

### Cellular Identification of LacZ-expressing Cells

Tie-2/LacZ/BMT mice were sacrificed 1 and 4 weeks after MI. The explanted hearts were sliced in a bread-loaf fashion into 3-transverse sections from apex to base (base-, mid- and apex-portion). Mid-portions of the hearts were fixed in 4% paraformaldehyde for 3 hours at room temperature and incubated in X-gal solution overnight at 37° C. The target tissue samples were then placed in PBS and examined under a dissecting microscope to detect sites of LacZ-expressing cells macroscopically. Also, histological sections were counterstained with nuclear fast red under 40× magnification and counted X-gal positive cells (blue stained cells) per sample in a blinded manner.

In addition, fluorescent immunohistochemical staining was performed using antibodies prepared against the rabbit anti-beta-galactosidase IgG (CORTEX Biochem) and the murine-specific EC marker, isolectin B4 (Vector Laboratories).

### EXAMPLE 9

### Reverse Transcripted PCR

Tissue RNA from frozen samples of injection sites of MI hearts (day 7) was extracted using TRIzol reagent (Invitrogen) according to the manufacturer's instructions. Reverse transcriptase (RT)-PCR for Gli-1, vascular endothelial growth factor (VEGF), angiopoietin-1, -2 Insulin growth factor-1 (IGF-1), stromal derived factor-1α (SDF-1α), and GAPDH genes was performed using 1 μg of total RNA. Amplification was carried out in 20 μL reaction mixtures containing 0.4 U Taq Polymerase (Clontech). Each cycle of PCR condition was 94° C. (30 s) and 64° C. (90 s). The following primer pairs were used for RT-PCR. Gli-1 (386 bp):

5′-CGGGTCTCAAACTGCCCAGCTT-3′
and
5′-GGCTGGGTCACTGGCCCTC-3′ with 32 cycles;
VEGF: 5′-ACCTCCACCATGCCAAGT-3′
and
5′-TAGTTCCCGAAACCCTGA-3′ with 30 cycles;
Angiopoietin-1 (169 bp):
5′-AGTCGGAGATGGCCCAGATACAACA-3′
and
5′-TCCAGCAGTTGGATTTCAAGACGGG-3′ with 37 cycles;
Angiopoietin-2 (259 bp):
5′-TACGTGCTGAAGATCCAGCTGAAGG-3′
and
5′-AGTTGGAAGGACCACATGCGTCGAA-3′ with 37 cycles;
IGF-1 (189 bp): 5′-ACTTCAACAAGCCCACAGGCTA-3′
and
5′-TCCTTCTGAGTCTTGGGCATGT-3′ with 32 cycles;
SDF-1α (223 bp): 5′-ATGGGACGCCAAGGTCGTCG-3′
and
5′-TCGGGTCAATGCACACTTGTCTGT-3′ with 32 cycle.

### EXAMPLE 10

### Primary Cultured Adult Cardiac Fibroblasts

To examine the direct effect of SHh on the cardiac fibroblasts, adult cardiac fibroblasts were prepared by selective attachment as described by Crabos and colleagues {Crabos, 1994}. Briefly, adult male Sprague-Dawley rats (200-250 g) was decapitated and cleaned with ethanol before the thorax will be incised. Hearts were excised, minced, and washed in PBS (GIBCO) supplemented with antibiotics (penicillin 100 U/ml and streptomycin 100 mg/ml; GIBCO). The tissue was then incubated at 37° C. for 20 min with 0.1% trypsin (Sigma Chemical, St. Louis, Mo.), 0.1% collagenase (Type CLS, Worthington Biochemicals), and 0.1% BSA (Sigma Chemical). Dissociated cells were centrifuged (for 5 min at 1,000 rpm) at the end of each of several incubation periods, and the cell pellet was resuspended in DMEM (GIBCO) supplemented with 10% fetal bovine serum (FBS; GIBCO) and antibiotics. Cells were then seeded into a 25-cm² flask (Nucon; GIBCO) and incubated at 37° C. in a humidified atmosphere of 5% CO₂-95% air. After 2 h, unattached cells and debris were discarded, and attached cells (nonmuscle cells, mostly fibroblasts) were cultured further in DMEM containing 10% FBS. This procedure yield cultures of cells that are almost exclusively fibroblasts by first passage, as determined by immunocytochemical techniques. For SHh treatments, cells were then passaged with trypsin (0.25%), plated in non-coating 35 mm plates at a density of 8-10,000 cells/cm2 in DMEM+10% FBS for 24 h. SHh protein will be supplemented at the appropriate concentration (1, 5, 10 μg/ML) in serum free DMEM+antibiotics supplement. Cells were harvested after 48 h with or without SHh treatment and RNA was extracted using RNA queous (Ambion) and RT-PCR was performed described above.

### Discussion

There have been reports that exogenous administration of Sonic hedgehog (SHh) acts on interstitial mesenchymal cells (such as fibroblasts) and regulates the secretion of various cytokines to promote functional recovery in animal models of hindlimb ischemia, for instance. According to the present invention, myocardial injection of human SHh plasmid is believed to promote a favorable effect on recovery of acute myocardial ischemia (MI).

Expression of SHh Protein in the Transfected Cell and Media

To examine the transgene effect of phSHh, phSHh was transfected into COS-1 cells and verified protein expression. See the Examples above. It was shown that immunoblotting for SHh protein. Twenty-four hours after transfection of phSHh to COS-1 cells, the expression of SHh protein was observed in both cell lysate and media indicating that this construct (phSHh) can result in secretion of SHh protein from cells. After adding the supernatant from transfected cells to the culture medium of nerve fibroblasts, and increase in Gli-1 expression, which is downstream target gene for SHh, was observed after 48 h. These data verify intact biologic activity of SHh protein secreted from cells transfected with this construct.

More specifically, expression of SHh protein and Gli mRNA was shown by imunoblotting. This revealed that 24 hours after transfection of plasmid of human sonic hedgehog (phSHh) into COS-1 cell. SHh protein was observed in both cell-lysate and medium but LacZ plasmid did not induce SHh protein expression. To verify the biological activity of phSHh-media, 24 hours after transfection of phSHh, conditioned medium was added to cultured nerve fibroblasts. 48 hours after incubation, Gli-1 expression was observed in phSHh treated media, indicating biologic activity of the SHfh protein secreted from cells transfected with this construct.

phSHh Preserves LV Function after Myocardial Ischemia

LV function was evaluated by echocardiography 5 days after LAD ligation. LVDd, LVDs, fractional shortening (FS), and regional wall motion score were similar among the five treatment groups. See the Examples above. From two to four weeks after MI, LVDd and FS worsened in rats receiving PBS, pLacZ and SHh 30 □g. In contrast, both parameters were preserved in SHh-300 and 600 μg groups: 4 weeks after induction of myocardial ischemia, LVDd was significantly smaller (PBS: 1.11±0.02, pLacZ: 1.12±0.02, phSHh-30 □μg 1.08±0.02, phSHh-300 μg 1.00±0.02, phSHh-600 μg 1.00±0.02 mm); FS was significantly greater (PBS: 18.6±0.4, pLacZ: 17.3±0.5, phSHh-30 μg 18.3±0.5, phSHh-300 μg 23.0±0.5, phSHh-600 μg 22.9±0.5%) and the change of regional wall motion score (pre minus post) was significantly better (PBS: −1.8±0.6, pLacZ: −2.7±0.6, phSHh-30 μg −2.2±0.6, phSHh-300 μg 3.7±0.8, phSHh-600 μg 3.8±0.8) in the phSHh-300 and 600 μg groups, compared with pLacZ group (P<0.01). LVDd, FS and regional wall motion score were similar in phSHh-300 and 600 μg groups.

Hemodynamic measurements were performed 4 weeks after MI. There was no significant difference in LV systolic pressure and LV end-diastolic pressure among the treatment groups. However, LV developed pressure (systolic pressure minus end-diastolic pressure) was significantly greater in phSHh-300 and 600 ug groups (PBS: 82.1±0.9, pLacZ: 82.2±2.3, phSHh-300 μg 82.6±1, phSHh-300 μg 88.6±2.8, phSHh-600 μg 88.3±1.2 mmHg, P<0.05 respectively). In addition, both LV+dP/dt and −dP/dt 4 weeks after MI were significantly better in phSHh-300 μg (+dP/dt: 3750±95, −dP/dt: −2516±90 mmHg/sec) and 600 μg (+dP/dt: 3885±91, −dP/dt: −2514±71 mmHg/sec) groups as compared with those in PBS (+dP/dt: 3383±79, −dP/dt: −2125±85 mmHg/sec), pLacZ (+dP/dt: 3328±72, −dP/dt: −2214±39 mmHg/sec) and SHh 30 μg (+dP/dt: 3338±63, −dP/dt: −2250±29 mmHg/sec) group (P<0.01 respectively).

More specifically, physiological and hemodynamic assessment was undertaken as follows. Echocardiographic parameters at baseline (day5), 2 weeks (day4) and 4 weeks (day28) after phSHh injection in rats with myocardial ischemia. Left ventricular diastolic dimension (LVDd), fractional shortening (FS) and changes of wall motion score (post minus pre) were all improved in phSHh-300 and 600 μg group compared to controls. Hemodynamic measurements 4 weeks after MI: Left ventricular developed pressure were greater in SHh-300 and 600 μg group. Left ventricular maximum positive (+dP/dt) and negative (−dP/dt) pressure development were also greater in phSHh-300 and 600 μg group.

phSHh Enhances Neovascularization and Inhibits Myocardial Fibrosis

In the rat study discussed above, capillary density was significantly greater in the phSHh-300 μg (536±37/mm²) and 600 μg (541±25/mm²) groups than that in the PBS (351±18/mm²), pLacZ (343±9/mm²) and phSHh-30 μg group (351±18/mm²) (P<0.01 respectively). Capillary density in phSHh-300 and 600 μg groups was not significantly different. Elastic tissue-trichrome staining was performed to identify LV fibrosis after myocardial ischemia. The fibrotic area was significantly smaller phSHh-300 μg (17±1%) and phSHh-600 μg (18±1%) groups than in the PBS (26±3%), pLacZ (25±2%) and phSHh-30 μg (28±3%) group (P<0.01).

More specifically, we prepared representative immunohistochemical findings for isolectin B4 in ischemic myocardium of rats 4 weeks after phSHh injection. Capillary density in rat ischemic myocardium 4 weeks after phSHh injection was performed. Also, representative elastic-trichrome stained sections from rats 4 weeks after phSHh injection were prepared. Left ventricular fibrosis was significantly reduced in rats with phSHh-300 and 600 μg group.

phSHh Enhanced Contribution of BM Derived EPCs to Myocardial Neovascularization

To examine whether phSHh injection augments recruitment of bone-marrow derived endothelial progenitor cells (EPC) into sites of myocardial neovascularization, Tie-2/lacZ/BMT mice were sacrificed 1 and 4 weeks after MI. See the Example above. It was shown that X-gal staining was more evident in the ischemic area of the phSHh injected heart 1 week after MI compared with the PBS injected heart. Four weeks after MI, the blue area in the PBS injected heart had almost disappeared, X-gal staining was still observed in the phSHh injected heart. Microscopic examination revealed that the number of X-gal positive cells in the phSHh group, at both 1 and 4 weeks after MI, was significantly greater as compared with the PBS group (1 wk: 302±31 versus 119±15/mm², P<0.01, 4 wks: 58±9 versus 19±5/mm², P<0.01;). Furthermore, fluorescent immunohistochemistry performed on paraffin-sections 1 week after MI showed that 62-gal positive cells were co-localized with isolectin-B4 positive cells in the ischemic area, indicating this β-gal positive cells differentiate into endothelial cells.

More specifically, enhanced contribution of BM-derived EPCs to myocardial neovascularization was observed. Representative macroscopic photographs were taken following X-gal staining of myocardium in Tie-2/LacZ/BMT mice 1 and 4 weeks after MI. X-gal was more evident in the ischemic area in the phSHh injected heart 1 week after MI compared with PBS injected heart. Four weeks after MI, evidence of X-gal positivity in the PBS injected heart was virtually absent, however weak X-gal expression was still observed in the phSHh injected heart. Representative photomicrographs following X-gal staining of myocardium in Tie-2/LacZ/BMT mice were also made. X-gal positive cells 1 week after MI. X-gal positive cells indicate Tie-2-expressing BM-derived EPCs. Quantitative analyses of X-gal positive cells between SHh and control groups 1 and 4 weeks after MI was performed. Also, representative photomicrographs were obtained after immunofluorescent histochemistry examination of myocardium in neovascularization assay of Tie-2/LacZ/BMT mice 1 week after MI. Isolectin B4 binding cells and β-gal binding cells were seen. Double positive cells were also seen indicating BM-derived EPCs incorporated into the neovasculature.

RT-PCR

To identify the potential mechanisms responsible for the therapeutic effect of SHh, we evaluated the expression of the Hh related transcriptional factor Gli-1 and certain angiogenic cytokines (VEGF-1, angiopoietin-1 and 2), anti-apoptotic cytokine (IGF-1) and trafficking chemokines for hematopoietic stem cells (SDF-1α) in treated (phSHh-300 μg) and control (pLacZ 300 μg) rat hearts harvested at day 7. See the Examples above. It was shown that endogenous Gli-1 expression was not observed in control rat heart, suggesting that the Hh pathway was inactivated under baseline conditions. However phSHh injection resulted in a significant increase in Gli-1 mRNA expression as well as upregulation of VEGF, angiopoietin-1 & 2, insulin-like growth factor (IGF) and SDF-1α. To verify these findings and to establish a direct effect of SHh on gene expression, RT-PCR was repeated on primary cultured adult cardiac fibroblasts. Expression of angiopoietin-1 and 2 was not detected in the cultured fibroblasts. However the expression of mRNA for all cytokines (VEGF, angiopoietin-1 and 2, IGF,) as well as Gli-1 was upregulated by SHh protein in a dose dependent manner (1, 5, 10 μg/ML), suggesting that SHh stimulation of cardiac fibroblasts can modulate expression of multiple factors with the potential to promote favorable functional recovery after MI.

More specifically, expression of mRNA in MI and peri-infarct tissue and primary cultured adult fibroblasts was seen. Representative examples of RT-PCR was conducted: phSHh injection upregulates transcription factor Gli, VEGF, angiopoietin-1 and -2, insulin-like growth factor-1 (IGF-1), SDF-1α but downregulates metaroproteinase-2 and -9. Additional representative examples of RT-PCR were performed: Sonic hedgehog protein supplementation of culture medium (1, 5, 10 μg/ML). upregulates Gli, VEGF, angiopoietin-1 and -2, IGF-1, SDF-1α but downregulates metaroproteinase-2 and -9 in a dose-dependent manner.

The Examples show that SHh gene therapy enhances neovascularization and prevents fibrosis and enhances functional recovery after acute myocardial ischemia. Without wishing to be bound to theory, this effect involves upregulation of multiple endogenous angiogenic cytokines as well as enhancement of bone-marrow derived EPC recruitment, In addition, it was also found that the upregulation of endogenous anti-apoptotic cytokines and trafficking chemokines for hematopoietic stem cells. These multiple favorable mechanisms are consistent with a reduction in the extent of LV scarring and enhanced preservation of LV function in the chronic phase after MI. Thus, local intramyocardial administration of a single gene, encoding for a secretable morphogen, in the peri-infarct period will exert favorable effects associated with multiple mono-therapies i.e angiogenesis, cell therapy and anti-apoptosis.

Patients who sustain large myocardial infarctions are at high risk of developing heart failure, even if they escape acute-phase death. The invention provides a new means of preserving or restoring myocardial function post-MI would be a highly desirable adjunct to existing therapies.

Gli-1 expression was examined as a marker for activation/inactivation of Hh/Ptc/Gli pathway. After pLacZ injection there were no activation of Hh pathway, indicating that during myocardial ischemia, the Hh pathway is not endogenously activated. However upregulation of Gli expression was found after injection of phSHh. These findings are consistent with previous SHh protein administration.

The results reported herein show that phSHh preserves LV function and prevents fibrosis. More specifically, the functional and histological data revealed that marked improvements were observed after phSHh injection. Surprisingly only 2 weeks after phSHh gene injection, a significant difference in the LV diameter, indicating prevention of the chronic remodeling had occurred in the early phase post MI was found. A low dose of SHh (30 μg) did not affect any detectable parameters for cardiac function, establishing a dose-response relationship and thereby providing important support for the true biological nature of the observed resposnes. Histological data revealed that capillary density was much higher in the mid to high dose phSHh groups compared with control and low dose phSHh-30 μg groups. In addition it appeared that more mature vessels (large and thicker vessels) were observed in the phSHh-300 and 600 μg groups. These observations are consistent with previous reports, in hind-limb ischemia {Pola, 2001}, for instance. We also found a marked reduction (about 30%) of fibrosis area 4 weeks after phSHh injection. These findings provide a new therapeutic strategy to prevent progressive heart failure after myocardial ischemia.

The Examples also show that phSHh modulates enhancement of neocapillaries. Specifically, the data show that SHh promotes neovascularization in the ischemic borderzone via upregulation of multiple angiogenic cytokines (VEGF, angiopoietin-1 and -2). Without wishing to be bound to theory, the effect result from SHh/ptc1/Gli signaling specifically in mesenchymal cells (fibroblasts) as shown previously via SHh protein administration {Pola, 2001}. In the foregoing examples, it was also shown that enhanced recruitment of bone-marrow (BM) derived endothelial progenitor cells (EPCs) as an additional mechanism for SHH mediated myocardial protection. The present disclosure shows that the upregulation of SDF-1α in the ischemic heart tissue following SHh gene therapy is associated with enhanced recruitment and incorporation of BM derived EPCs in the ischemic borderzone.

The present disclosure also shows that phSHh upregulates antiapoptoic cytokines from fibroblasts. There have been reports that the fibroblast is a major cell type within the myocardium and plays a critical role in the composition and structure of the heart. It is well known that changes in myocardial fibroblast form and function contribute significantly to the remodeling process after MI. It was found that upregulation of IGF-1 mRNA in ischemic hearts followed phSHh gene therapy and in SHh treated cultured cardiac fibroblasts. Without wishing to be bound to theory, the data suggest that SHh induced IGF-1 may also contribute to the preservation of myocardial integrity post-MI.

Without wishing to be bound to theory, the present disclosure is consistent with local, intramyocardial SHh gene therapy triggering a cascade of downstream trophic factors that can enhance recovery following MI via activation of multiple targets. SHh gene therapy will offer significant advantages over approaches employing a single factor. These data are also show that intramyocardial SHh gene delivery preserves chronic LV function after MI by upregulation of various endogenous angiogenic and anti-remodeling cytokines. This “multifactor” approach is believed to provide a significant advantage to enhance recovery from MI as compared to mono-cytokine gene therapy.

The following Examples are further illustrative but not limiting of the present invention.

### EXAMPLE 11

### Post-Natal Activation of Shh Signaling Pathway in the Ischemic Myocardium

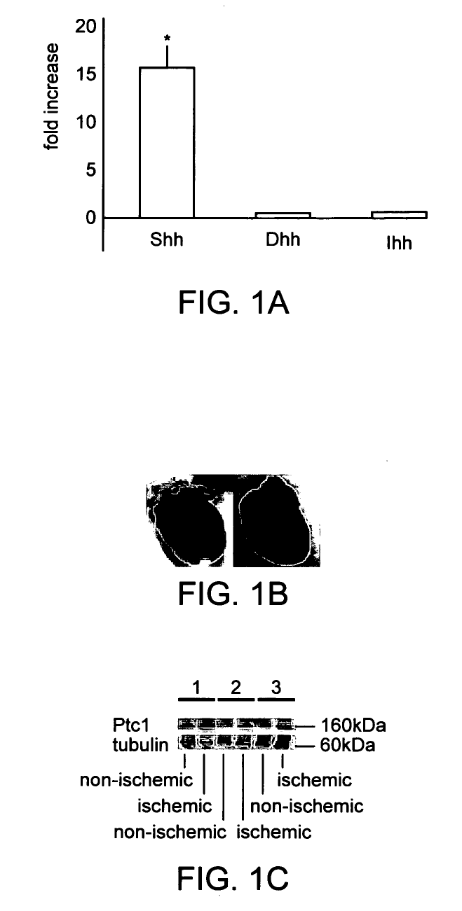

Real time RT-PCR for Shh, Dhh and Ihh performed 4 days after the onset of myocardial ischemia in a mouse LAD ligation model revealed that Shh mRNA expression was increased 15±3.1 fold in ischemic myocardium (FIG. 1A, P<0.01). No significant increment of Dhh and Ihh expression was documented.

The expression of the hedgehog receptor Ptc1 was examined. Ptc1 expression by β-galactosidase (β-gal) staining of heart tissues of mice was analyzed that carrys a mutation of one allele of the Ptc1 gene consisting of insertion of a lacZ reporter gene upstream of the ptch coding region (Ptc1-lacZ mice).²¹ Seven days after myocardial ischemia, whole-mount X-gal staining shows strong upregulation of Ptc1 in the ischemic border zone myocardium (FIG. 1B). FIG. 1C shows a representative Western Blot for Ptc1 seven (7) days after myocardial infarction. The Ptc1 signal is increased in the ischemic myocardium, compared to non-ischemic myocardium. The data shows that the Shh pathway is intact post-naturally and is functional in the heart in the setting of MI (myocardial infarct). The Shh receptor Ptc1 is expressed not only in cardiac fibroblasts but also in cardiomyocytes, a previously undisclosed finding.

FIGS. 1A-C are explained in more detail as follows. A: Real-time RT-PCR 4 days after ischemia shows that Shh mRNA is increased 15±3.1 fold (P<0.01, n=3 per group) in the ischemic myocardium compared to the non-ischemic tissues, while Dhh and Ihh gene expression are not substantially altered. B: Ptc1 is upregulated after myocardial ischemia. Ligation of the anterior descending coronary artery was performed in NLS-Ptc1-lacZ mice. Compared with the control heart, whole mount X-gal staining seven days after myocardial ischemia shows strong upregulation for Ptc1 in the area surrounding the ischemic myocardium.C: Western blot analysis of Ptc1 expression in ischemic and non-ischemic myocardium. Marked Ptc1 upregulation was observed in the ischemic tissue.

### EXAMPLE 12

### Injection of Human Shh Plasmid Upregulates Shh Signaling Pathway

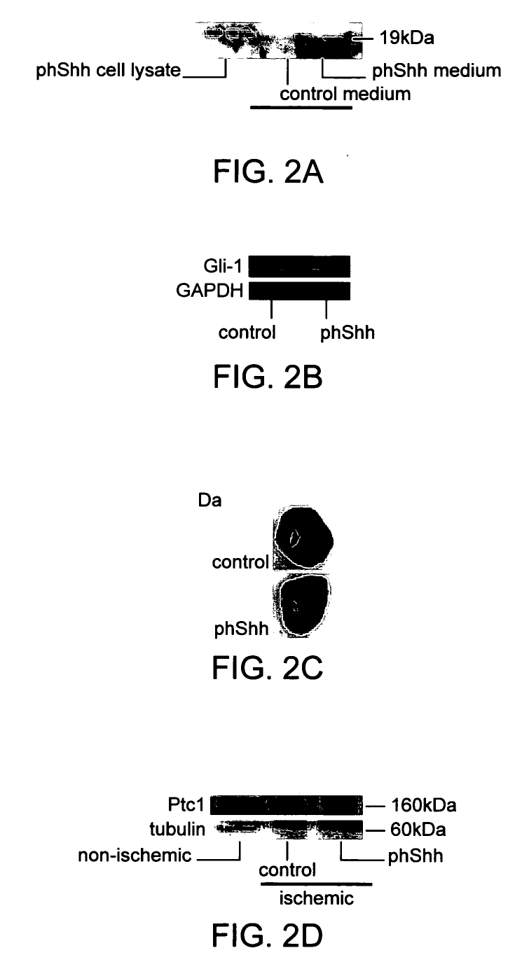

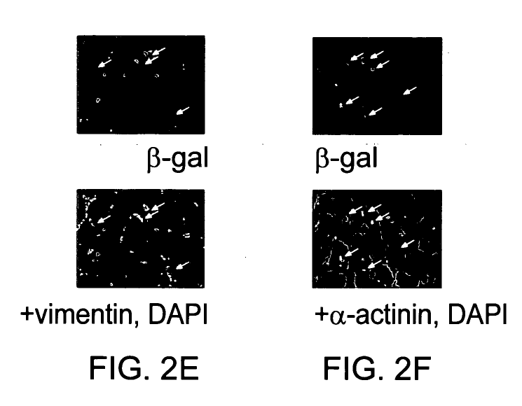

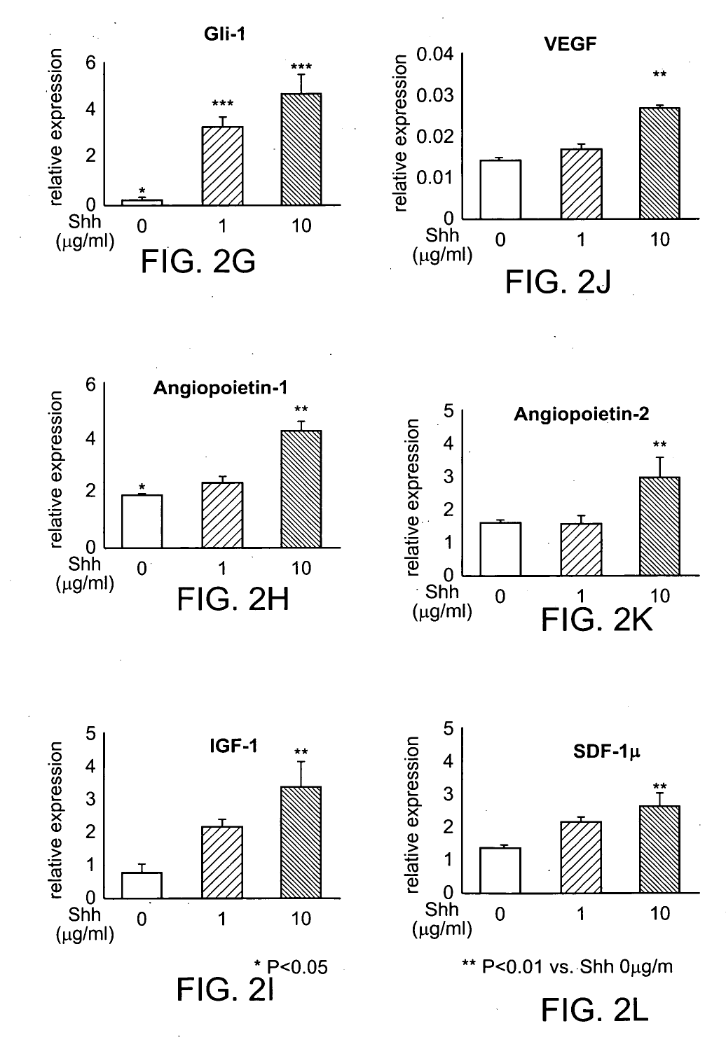

Seven days after LAD ligation and phShh injection, whole-mount X-gal staining (FIG. 2C) showed stronger expression of Ptc1 in NLS-Ptc1-lacZ mice. Western blots verified that Ptc1 expression induced by ischemia was further upregulated by overexpression of Shh (FIG. 2D). Immunofluorescent staining for β-gal, performed in NLS-Ptc1-lacZ mice, demonstrated the expression of Ptc1 in several cells in both ischemic and non-ischemic tissue. By performing double immunofluorescence analysis, for β-gal and vimentin in NLS-Ptc1-lacZ mice, several interstitial cells show expression of both markers, indicating that cardiac fibroblasts express Ptc1 as previously reported (FIG. 2E). This analysis also revealed that a-actinin-positive cells also express Ptc1 indicating cardiomyocyte expression of these receptors (FIG. 2F).

FIGS. 2A-F are explained in more detail as follows: A and B show expression of Shh protein and Hh-related transcriptional factor Gli mRNA, respectively, after plasmid transfection. Immunoblotting revealed that 24 h after transfection of phShh into COS cells, Shh protein was observed in both cell lysate and medium but LacZ plasmid did not induce Shh protein expression (A). To verify the biological activity of phShh-medium, 24 h after transfection of phShh, conditioned medium was added to primary cultured cardiac fibroblasts. 48 h after incubation, Gli-1 expression was observed in phShh treated medium, indicating biologic activity of the Shh protein secreted from cells transfected with this construct (B). C: Ptc1 is upregulated after phShh injection in NLS-Ptc1-lacZ mice. Whole mount X-gal staining seven days after plasmid injection revealed strong upregulation for Ptc1 in the ischemic area. D: Representative Western blotting for Ptc1 seven days after plasmid injection. Further upregulation of Ptc1 was observed in phShh mice tissue.

### EXAMPLE 13

### phShh Upregulates Multiple Cytokines in Cardiac Fibroblasts

To identify the potential mechanisms responsible for the therapeutic effect of Shh, the mRNA expression of a panel of candidate genes in primary cultured cardiac fibroblasts was evaluated (FIG. 2G-L). FIGS. 2E-L are explained in further detail as follows: E,F show double immunostaining in NLS-Ptc1-lacZ mouse myocardium after phShh injection. Immunofluorescence staining shows that β-gal positive cells (red), colocalized with vimentin (green in d) and α-actinin (green in E), revealing that both fibroblasts and cardiomyocytes express the Shh receptor Ptc1; G-L: show quantitative RT-PCR in primary cultured adult fibroblasts. Sonic hedgehog protein supplementation of culture medium (0, 1, 10 □g/ml) upregulates Gli-1, VEGF, angiopoietin-1, and -2, IGF-1, and SDF-1\tilde{□} Data are expressed as mean±SE. Each experiment was repeated at least 4 times.

As discussed, a dose dependent upregulation of the Hh related transcription factor Gli-1 was observed, reiterating that the hedgehog pathway is intact in these cells. A variety of angiogenic cytokines were upregulated by Shh, including VEGF, angiopoietin-1 and 2. However it was found that IGF-1, a key survival and growth factor for cardiomyocytes, was significantly upregulated. In addition, the expression of SDF-1α, a trafficking chemokine for hematopoietic stem cells, was also increased. These findings are consistent with earlier studies documenting the ability of Shh to promote neovascularization, but also suggested that in the myocardium, Shh is promoting other effects. For example, the finding of increased SDF-1α expression, led us to consider function involving recruitment of bone marrow derived progenitor or stem cells. This is a mechanism for the rapid effects required for tissue preservation in the acute infarct model.

Thus, the Example shows that phShh upregulated multiple cytokines from cardiac fibroblasts. In particular, the fibroblast is the most abundant cell type within the myocardium and plays a critical role in the composition and structure of the heart. It has been reported that Shh acts on mesenchymal cells and induces the secretion of multiple angiogenic cytokines including VEGF and angiopoietins. The Example confirmed these results, while also demonstrating that the anti-apoptotic cytokine IGF-1 and the trafficking chemokine for hematopoietic stem cell, SDF-1α were also upregulated.

### EXAMPLE 14

### phShh Inhibits Myocardial Fibrosis and Enhances Neovascularization

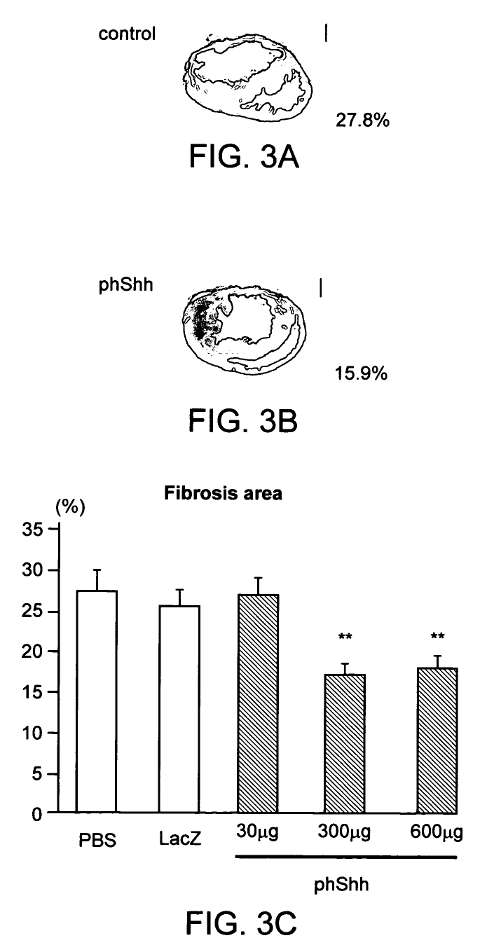

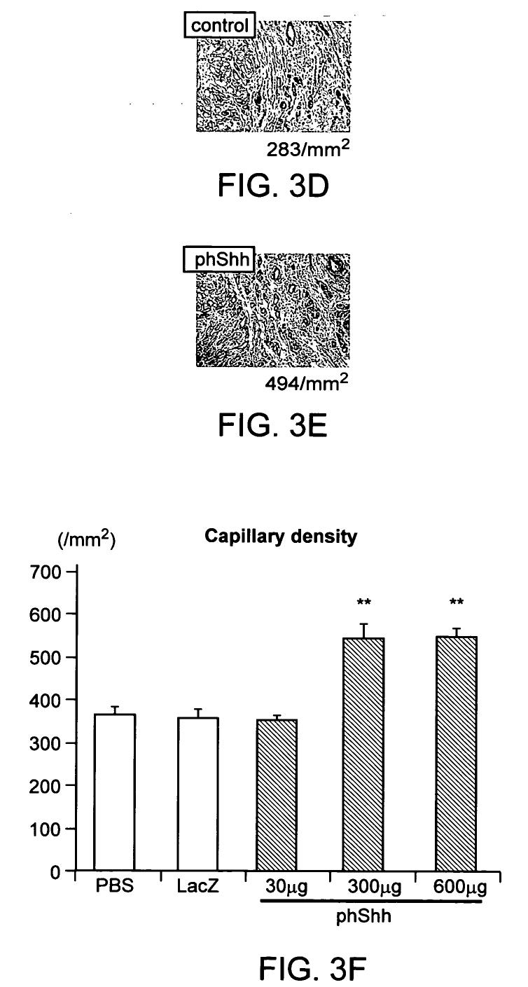

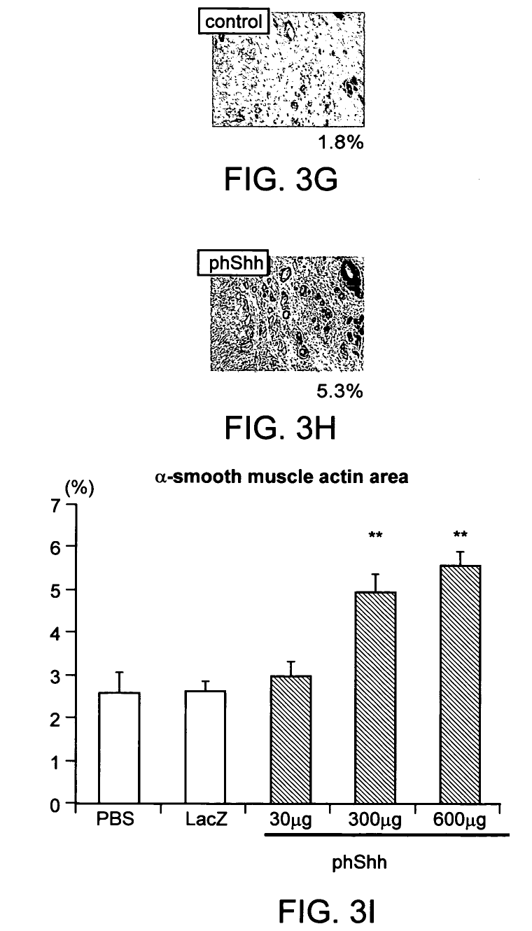

Having established that the Shh was intact in the myocardium and that the expression vector produced functional secreted protein, work was started in models of acute and chronic myocardial ischemia. In the rat acute myocardial infarction model, elastic tissue-trichrome (ET) staining was performed to identify LV fibrosis following infarction. The fibrotic area was significantly smaller in the phShh-300 μg (17±1%) and phShh-600 μg (18±1%) groups than in the PBS (26±3%), lacZ plasmid (25±2%) and phShh-30 μg (28±3%) groups (P<0.01, FIG. 3A-C). Capillary density was significantly greater in the phShh-300 μg (536±37/mm²) and 600 μg (541±25/mm²) groups than that in the PBS (351±18/mm²), lacZ plasmid (343±9/mm²) and phShh-30 μg groups (351±18/mm²) (P<0.01, FIG. 3D-F). The total smooth muscle area in the ischemic zone was assessed. The α-smooth muscle acting positive area was significantly greater in the phShh-300 μg (4.88±0.31%) and 600 μg (5.54±0.31%) groups than that in the PBS (2.50±0.49%), LacZ plasmid (2.58±0.20%) and phShh-30 μg groups (2.93±0.37%) (P<0.01, FIG. 3G-I).

FIGS. 3A-I are described in further detail as follows: A-C show anatomic assessment after myocardial infarction in rat. A,B: representative elastic-trichrome stained sections from rats 4 weeks after phShh vs. control plasmid injection; C: left ventricular fibrosis was significantly reduced in rats with phShh-300 and 600 μg group; D-F: representative immunohistochemical findings for isolectin B4 in ischemic myocardium of rats 4 weeks after phShh injection. Capillary density was significantly preserved in rats with phShh-300 and 600 μg group; 3G-I: representative immunohistochemical findings for a-smooth muscle acting. More smooth muscle acting positive vasculature was observed in phShh injected rat heart. Data were expressed as mean±SE. Control PBS, n=6, control LacZ plasmid, n=7, phShh-30 μg, n=6, phShh-300 μg, n=9, phShh-600 μg, n=8. **P<0.01 vs. lacZ plasmid.

### EXAMPLE 15

### phShh Preserved LV Function After Acute Myocardial Infarction (MI)

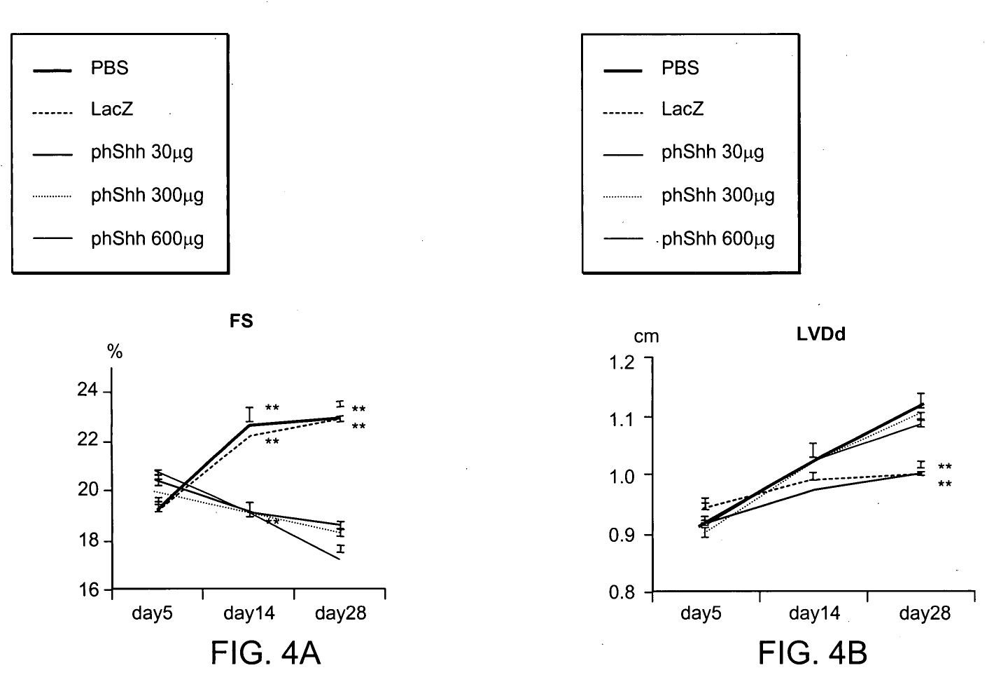

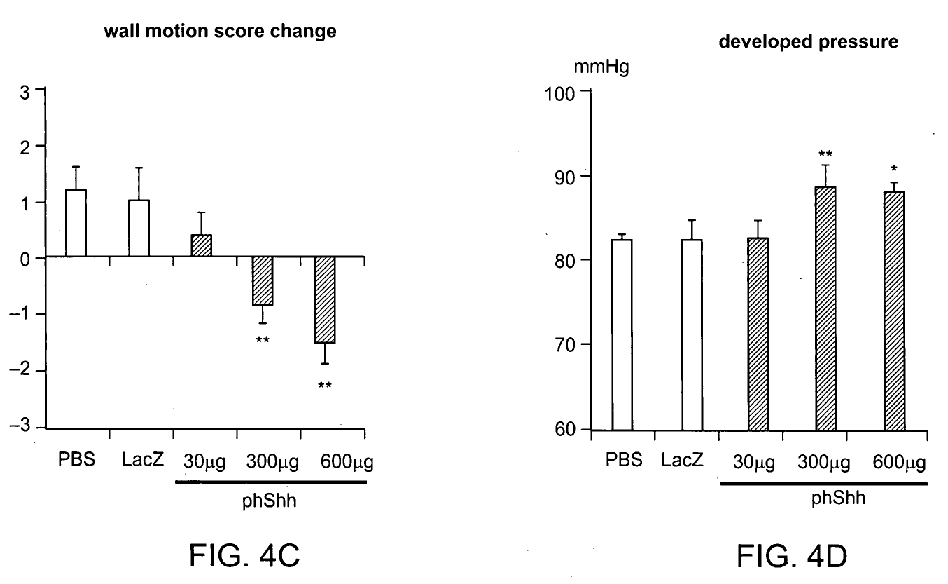

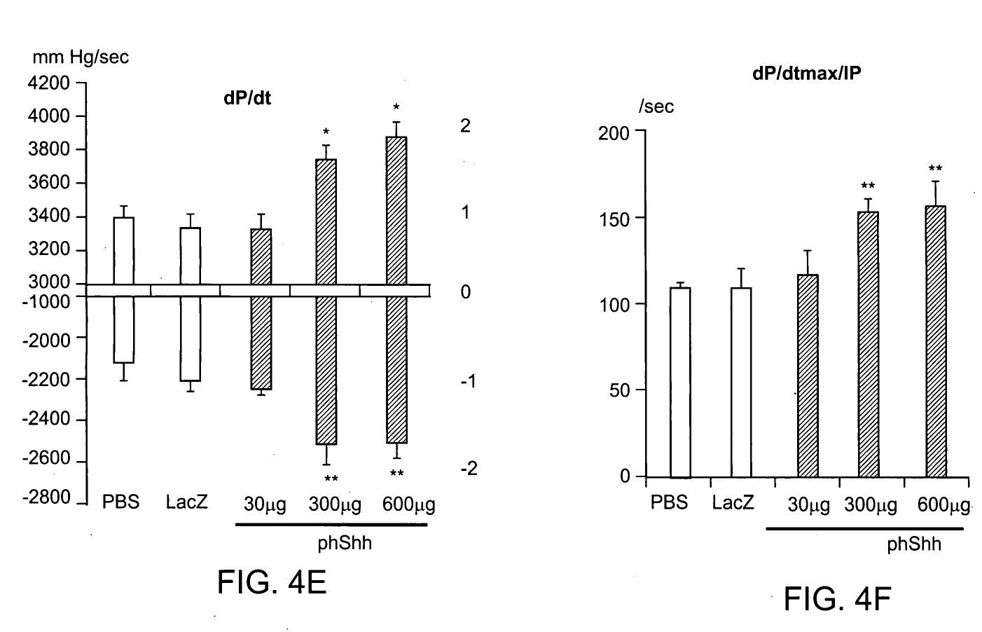

After MI, LV function has been shown to be one of the key indicators of overall prognosis and outcome. In general terms, the preservation of the ability of the heart muscle to contract in a coordinated fashion is a positive finding, while dilation of the LV chamber augurs a worse prognosis. Moreover, the serial changes in LV function and dimensions prove useful in portending long-term outcome, and for this purpose echocardiography, which uses ultrasound to examine these parameters, is the most widely employed technique. LV function was evaluated by echocardiography 5 days after LAD ligation. LVDd, LVDs (measures of cardiac dilation) and fractional shortening (FS), and regional wall motion scores (which determine contractile function) were similar among the five treatment groups. From two to four weeks after MI, LVDd and FS worsened in rats receiving PBS, control lacZ plasmid and phShh-30 μg. In contrast, both parameters were preserved in phShh-300 and 600 μg groups: 4 weeks after induction of myocardial ischemia, LVDd was significantly smaller (better) (PBS: 1.11±0.02, lacZ plasmid: 1.12±0.02, phShh-30 μg 1.08±0.02, phShh-300 □g 1.00±0.02, phShh-600 μg 1.00±0.02 mm); FS was significantly greater (PBS: 18.6±0.4, lacZ: 17.3±0.5, phShh-30 μg 18.3±0.5, phShh-300 μg 23.0±0.5, phShh-600 β g 22.9±0.5%) and the change of regional wall motion score (post minus pre) was significantly better (PBS: 1.8±0.6, lacZ plasmid: 2.7±0.6, phShh-30 μg 2.2±0.6, phShh-300 μg -3.7±0.8, phShh-600 μg −3.8±0.8) the phShh-300 and 600 μg groups, compared with other groups (P<0.01, respectively. FIGS. 4A-C). LVDd, FS and regional wall motion score were similar in phShh-300 and 600 μg groups.

Hemodynamic measurements can also provide accurate assessments of outcome and long-term prognosis after MI. The pressure generated by LV contraction, as well as the rate of pressure increase and relaxation all correspond with better LV function overall. Fours weeks after MI there was no significant difference in LV systolic pressure(maximum pressure at peak contraction) and LV end-diastolic pressure(Pressure in the LV at the end of the filling phase, just before the onset of LV contraction) among the treatment groups. However, LV developed pressure (systolic pressure minus end-diastolic pressure) was significantly greater (indicating better LV function) in phShh-300 and 600 μg groups (PBS: 82.1±0.9, lacZ plasmid: 82.2±2.3, phShh-300 μg: 82.6±2.1, phShh-300 μg: 88.6±2.8, phShh-600 μg: 88.3±1.2 mmHg, P<0.05 respectively, FIG. 4D). In addition, both LV+dP/dt and −dP/dt (the rate of contraction and relaxation) 4 weeks after MI were significantly better in phShh-300 μg (+dP/dt: 3750±95, −dP/dt: −2516±90 mmHg/sec) and phShh-600 μg (+dP/dt: 3885±91, −dP/dt: −2514±71 mmHg/sec) groups as compared with those in PBS (+dP/dt: 3383±79, −dP/dt: −2125±85 mmHg/sec), lacZ plasmid (+dP/dt: 3328±72, −dP/dt: −2214±39 mmHg/sec) and Shh 30 μg (+dP/dt: 3338±63, −dP/dt: −2250±29 mmHg/sec) groups (P<0.05 respectively, FIG. 4E). Similar results were disclosed using a relatively load-independent (felt to optimally represent true contractile function) systolic function index dP/dtmax/IP (instantaneous pressure). The dP/dtmax/IP 4 weeks after MI was significantly better in phShh-300 μg and 600 μg groups (PBS: 98.7±6.6, lacZ plasmid: 106.5±11.2, phShh-300 μg: 116.8±11.4, phShh-300 μg: 152.5±5.4, phShh-600 μg: 156.1±3.0/sec, P<0.01 respectively, FIG. 4F).

FIGS. 4A-F are described in more detail as follows: A-C: physiological and hemodynamic assessment after rat myocardial ischemia showing echocardiographic parameters at baseline (day 5), 2 weeks (day 14) and 4 weeks (day 28) after phShh injection in rats with myocardial ischemia. Left ventricular diastolic dimension (LVDd), fractional shortening (FS) and changes of wall motion score (post minus pre) were all restored in phShh-300 and 600 μg group compared to controls; 4D-F: hemodynamic measurements 4 weeks after MI: Left ventricular developed pressure was greater in Shh-300 and 600 μg group. Left ventricular maximum positive (+dP/dt) and negative (−dP/dt) pressure development and dP/dtmax/IP (instantaneous pressure) were also greater in phShh-300 and 600 μg group. Values were expressed as mean±SE. Control PBS, n=6, control LacZ plasmid, n=7, phShh-30 μg, n=6, phShh-300 μg, n=9, phShh-600 μg, n=8.* P<0.05, ** P<0.01 vs. lacZ plasmid.

The Example shows, among other things, that Shh gene transfer in the setting of acute myocardial infarction resulted in an improvement in myocardial function with reduced fibrosis and enhancement in myocardial performance and physiology.

### EXAMPLE 16

### phShh Reduced Chronic Myocardial Ischemia

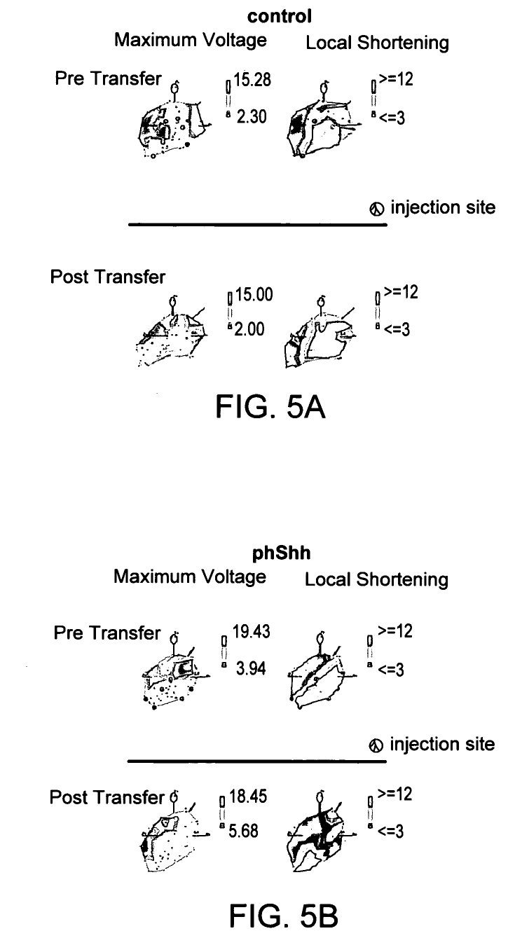

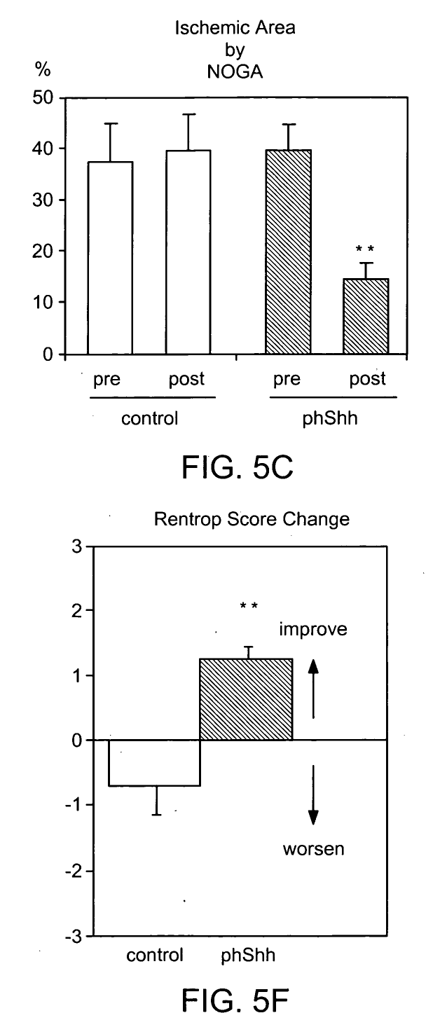

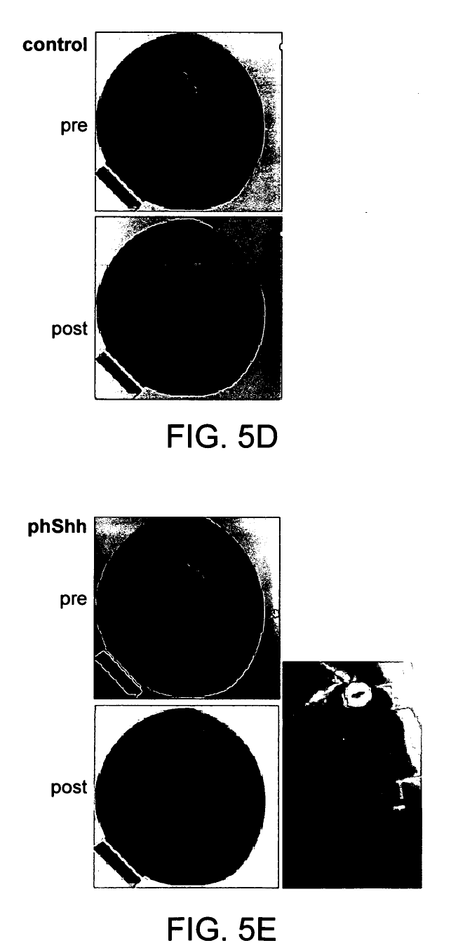

In the porcine model, a constrictor device is placed around the left circumflex artery which then slowly occludes the artery over the course of 5 weeks. This results in a zone of ischemia without the occurrence of acute myocardial infarction. The larger animal model also permitted the use of an endocardial mapping technology (NOGA) that is capable of quantifying myocardial ischemia in a real-time format. The ischemic area determined by NOGA mapping before gene transfer was not significantly different between control (lacZ plasmid) and phShh group (control: 37.5±7.3, phShh: 39.3±5.3%). Four weeks after treatment, the ischemic area was significantly reduced in phShh group (control: 39.7±7.1, phShh: 14.2±3.2%, P<0.01, FIG. 5A-C).

FIGS. 5A-C are described in further detail as follows: A,B: representative recordings of NOGA electromechanical mapping immediately before (pre transfer) and 4 weeks after (post transfer) plasmid injection. The dots in the pretreatment map show the sites of gene transfer. The red area on the pre transfer local shortening map indicates an area of decreased wall motion in the lateral wall of the left ventricle, consistent with ischemia in the territory of the left circumflex artery. Four weeks after gene transfer, this area of ischemia improved in a representative case in the phShh group (B), whereas no improvement was observed in case from control group (A); C: ischemic area pre and post gene transfer in each group. There is significant reduction in ischemic area in phShh group.

Table II, below, provides useful experimental information.

TABLE II
Effects of phSHh on echo parameters in the pig
model of chronic myocardial ischemia

| | Pre transfer | | Post transfer | |
|---|---|---|---|---|
| | Control | PhSHh | control | phSHh |
| LVDd (cm) | 4.89 ± 0.17 | 4.98 ± 0.11 | 5.60 ± 0.11 | 5.22 ± 0.85* |
| LVDs (cm) | 3.62 ± 0.15 | 3.71 ± 0.84 | 4.26 ± 0.11 | 3.62 ± 0.06** |
| Wall motion | 21.8 ± 0.30 | 22.2 ± 0.22 | 23.0 ± 0.46 | 20.4 ± 0.24** |
| score | | | | |
| Fractional | 26.2 ± 0.83 | 25.4 ± 0.47 | 24.0 ± 0.76 | 30.6 ± 0.42** |
| shortening | | | | |
| (%) | | | | |

Data are present as mean ± SE.
Abbreviations:
LVDd, left ventricular diastolic dimension;
LVDs, left ventricular systolic dimension; and
phSHh, plasmid of human sonic hedgehog
*P < 0.05,
**P < 0.01 vs. control

### EXAMPLE 17

### phShh Developed New Collaterals in the Chronic Myocardial Ischemia

Selective left coronary angiography was performed to evaluate collateral development before and after treatment. The mean Rentrop score of collateral development to the LCX territory at baseline was 1.2±0.2 in control group and 1.3±0.2 in phShh group (p=NS). However, after treatment, the mean Rentrop score was significantly improved in phShh group (2.4±0.2) compared with control plasmid group (0.5±0.2, P<0.01) indicating that Shh gene transfer had resulted in the development of macroscopic collateral vessels to the ischemic myocardium, thereby reducing myocardial ischemia (FIG. 5D-F).

FIGS. 5D-F are described in further detail as follows: D, E: representative findings of left coronary angiography pre and post-gene transfer. Abundant collateral blood vessels were observed to left circumflex artery; F: changes in Rentrop grade of collateral development 4 weeks after gene transfer. There is significant improvement in collateral development in phShh group. Data were expressed as mean±SE. Control LacZ plasmid, n=8, phShh, n=9.

### EXAMPLE 18

### phShh Improved Cardiac Function in the Chronic Myocardial Ischemia

Cardiac ultrasound examinations were preformed serially in all animals. Fractional shortening and regional wall motion scores before treatment were similar in both groups (FS: control; 26.2±0.8, phShh; 25.4±0.5%. Regional wall motion score: control; 21.8±0.3, phShh; 22.2±0.2, P=NS, respectively). Four weeks after gene transfer, however, both FS and regional wall motion score was significantly improved in the phShh group compared with the control plasmid group (FS: control; 24.0±0.8, phShh; 30.6±0.4%. Regional wall motion score: control; 23.0±0.5, phShh; 20.4±0.2, P<00.1 respectively, Table II).

Thus, two distinct in vivo models provided by the present examples, of acute and chronic myocardial ischemia, revealed that Shh gene transfer improved myocardial function associated with increases in myocardial perfusion and in both microscopic and macroscopic myocardial vascularization.

The Examples discussed herein show, among other things, that phShh promoted a favorable effect after acute and chronic myocardial ischemia. In particular, functional and histological data revealed that marked improvements were observed after phShh injection. Surprisingly, only 2 weeks after phShh gene injection, it was found that a significant difference in the LV diameter, indicating prevention of the chronic remodeling had occurred in the early phase post MI. A low dose of Shh (30 μg) did not affect any parameters for cardiac function, establishing a dose-response relationship and thereby providing important support for the true biological nature of the observed responses. Histological data revealed that capillary density was much higher in the mid to high dose phShh groups compared with control and low dose phShh-30 μg groups. In addition, it appeared that more mature vessels (smooth muscle marker, acting positive cells) were observed more in the phShh-300 and 600 μg groups. These observations were consistent with previous reports using recombinant Shh protein, in the hind-limb ischemia model²⁰. A marked reduction (about 30%) of fibrosis area was found 4 weeks after phShh injection. In the swine model of chronic ischemia, it was also found that marked reduction of ischemic area (66% reduction), development of new collaterals and preservation of LV performance after phShh injection. These findings suggest therapeutic utility to prevent progressive heart failure in the setting of severe myocardial ischemia.

### EXAMPLE 19

### phShh Enhanced Contribution of BM Derived EPCs to Myocardial Neovascularization

It was determined whether phShh injection augments recruitment of bone-marrow (BM) derived endothelial progenitor cells (EPC) into sites of myocardial ischemia. To do so, a chimeric mouse model was used. Tie-2/lacZ/BMT mice are the recipients of bone marrow from donors in which cells expressing the endothelial specific Tie-2 promoter express lacZ. This enables identification of cells of endothelial lineage that are derived from a bone marrow progenitor. After recovery from the bone marrow transplantation these mice were induced to undergo myocardial infarction, were randomly assigned to treatment with Shh plasmid or control plasmid (empty vector) and were sacrificed after 1 and 4 weeks.

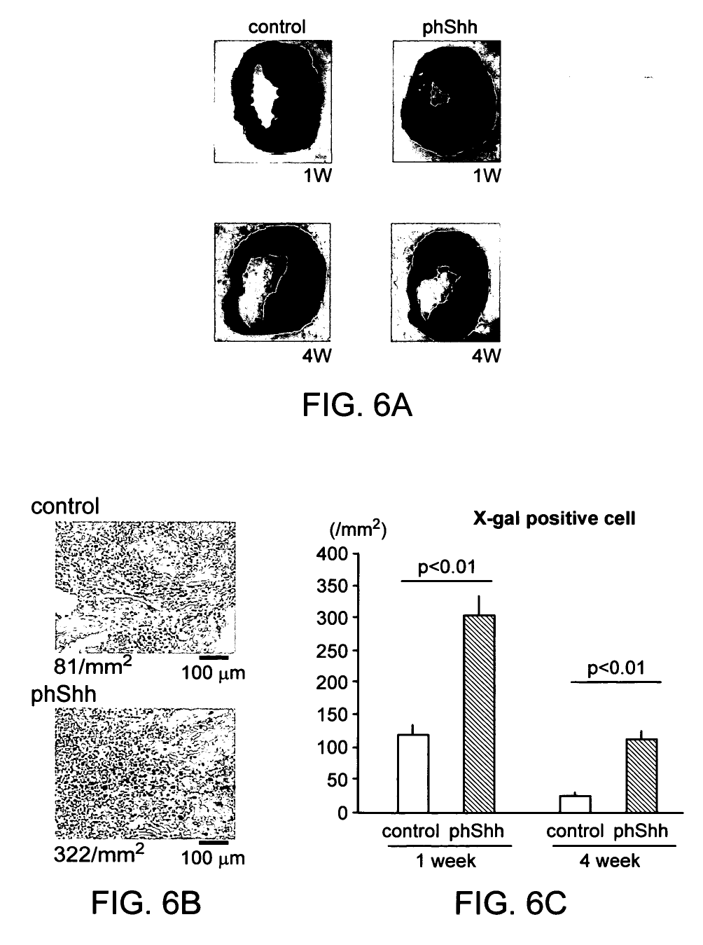

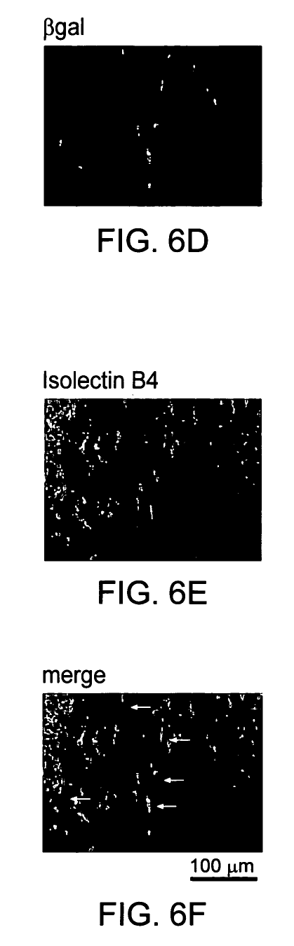

As shown in FIG. 6A, X-gal staining, which identified cells expression of lacZ, was more evident in the ischemic zones of the phShh treated hearts 1 week after MI compared with the control heart. Four weeks after MI, the blue, X-gal positive area in the control hearts had almost disappeared, while clear X-gal staining was still easily observed in the phShh injected hearts. Microscopic examination revealed that the number of X-gal positive cells in the phShh group, at both 1 and 4 weeks after MI, was significantly greater as compared with the control plasmid group (1 wk: control; 119±15; phShh 302±31/mm², P<0.01, 4 wks: control; 19±5, phShh; 58±9/mm², P<0.01, FIGS. 6B, 6C). Furthermore, fluorescent immunohistochemistry performed on paraffin-sections 1 week after MI showed that β-gal positive cells were co-localized with isolectin-B4 positive cells in the ischemic area (double staining, white arrows in FIG. 6D-F), providing confirmatory evidence that β-gal positive cells differentiate into endothelial cells in the myocardium.

FIGS. 6A-F are described in further detail as follows: A-C show contribution of bone marrow-derived endothelial progenitor cells (EPCs). A: representative macroscopic photograph following X-gal staining of myocardium in Tie-2/LacZ/BMT mice 1 and 4 weeks after MI. X-gal is more evident in the ischemic area in the phShh injected heart 1 week after MI compared with control. Four weeks after MI, evidence of X-gal positivity in the control heart was virtually absent, however weak X-gal expression was still observed in the phShh injected heart; B: representative photomicrographs following X-gal staining of myocardium in Tie-2/LacZ/BMT mice 1 week after MI. X-gal positive cells (blue) indicate Tie-2-expressing BM-derived EPCs; C: quantitative analyses of X-gal positive cells in Shh and control groups 1 and 4 weeks after MI reveals a significantly greater number of in X-gal positive cells in Shh treated hearts. N=5 per group.

Accordingly, the Example shows that phShh modulates enhancement EPC recruitment. In particular, it further shows that Shh promoted neovascularization in the ischemic borderzone. These effects may result from Shh/ptc1/Gli signaling specifically in mesenchymal cells (fibroblasts) as shown previously via recombinant Shh protein administration²⁰ leading to the upregulation of VEGF and angiopoietins. The Example further demonstrates enhanced recruitment of bone-marrow (BM) derived endothelial progenitor cells (EPCs) as an additional mechanism for Shh mediated myocardial protection. It has been shown that EPCs isolated from circulating mononuclear cells in peripheral blood incorporate into foci of neovascularization of adults, consistent with notion of postnatal vasculogenesis^{30,31}. These BM-derived EPCs are mobilized endogenously in response to tissue ischemia and/or exogenously by cytokine stimulation. Vascular endothelial growth factor (VEGF) and granulocyte macrophage-colony stimulating factor (GM-CSF) can mobilize EPCs from the BM into the peripheral circulation. On the other hand, stromal-derived factor-1α (SDF-1α) has been shown to act as a local recruitment factor for EPC¹⁰. The Examples show, among other things, that Shh induces the upregulation of SDF-1α a in adult cardiac fibroblasts and is associated with enhanced recruitment and incorporation of BM derived EPCs in the ischemic borderzone.

### EXAMPLE 20

### phShh Prevents Cardiac Apoptosis

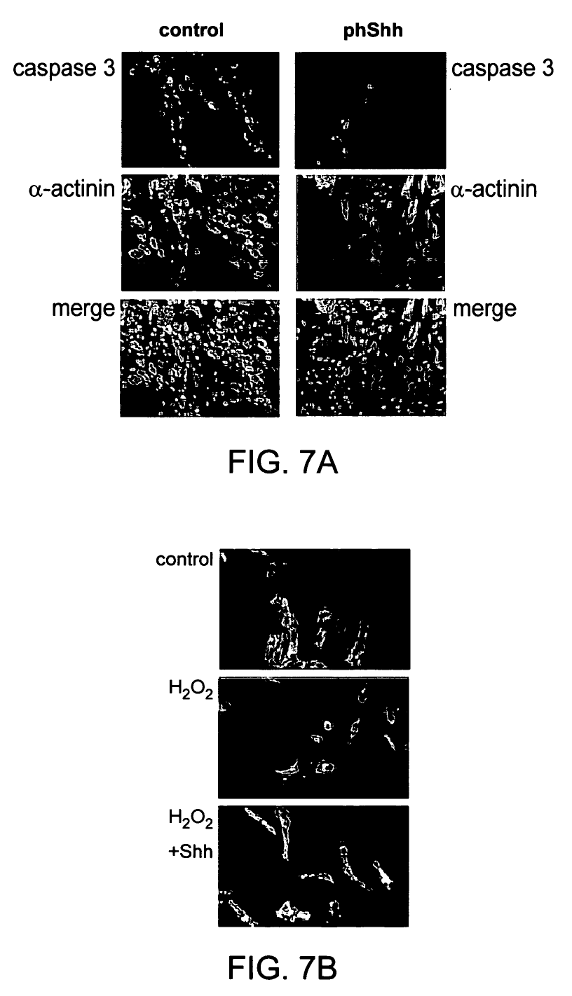

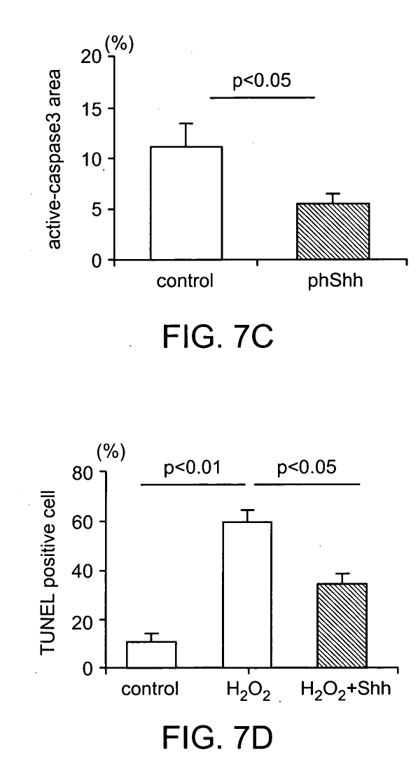

The finding that Shh gene therapy promotes recruitment of bone marrow derived progenitor cells to the myocardium suggested how the gene functions. In addition to incorporating into new vascular structures, these cells act as repositories of multiple cytokines that exert proliferative and survival effects. To examine the potential for an anti-apoptotic effect, we performed active-caspase3 staining 4 days post MI in mice (FIG. 7A). Compared with control plasmid, fewer active-caspase3 positive cardiomyocytes were observed in the myocardium of Shh gene treated mice (control: 11.2±2.1; phShh: 5.5±0.9% active-caspase3 area/total area, P<0.05, FIG. 7C).

To determine if Shh could directly exert the observed in vivo anti-apoptotic effect, we performed experiments on cultured neonatal rat cardiomyocytes (FIG. 7B). Apoptosis was induced by hydrogen peroxide (H₂O₂; 100 μM). Pretreatment with Shh protein significantly reduced the number of TUNNEL positive cardiomyocytes (control: 9.2±3.8, H₂O₂: 58.6±5.9, H₂O₂+Shh: 35.9±4.4%, P<0.01 between control and H₂O₂, P<0.05 between H₂O₂ alone and H₂O₂+Shh, FIG. 7D).

FIGS. 7A-D, which show that Shh reduces cardiomyocyte apoptosis in vivo and vitro are described in further detail as follows: A,C: activated-caspase3 staining in mouse myocardium four days after MI and plasmid injection. Fewer activated-caspase 3 positive cardiomyocytes (red&green) were noted in the phShh injected hearts compared with the control plasmid injected heart (FIG. 7C, N=5 per group); B:D Shh protects cardiomyocytes from H₂O₂-induced apoptosis. Cardiomyocytes were prepared from neonatal rats and stained with mouse anti □-sarcomeric actinin followed by rhodamine conjugated goat anti mouse IgG. Cardiomyocytes were incubated with H₂O₂ for 24 hours with or without recombinant human Shh protein (10 □/ml) for 48 hours. Shh protein significantly reduced H202 induced cardiomyocyte apoptosis. Data were obtained from 15 different fields (FIG. 7D).

Thus, the example shows that phShh promotes anti-apoptotic effect. It was observed that Shh upregulates the mRNA expression of IGF-1 associated with a reduction in area of active-caspase3 positive cardiomyocytes in the infarct zone. Furthermore, we demonstrated that Shh inhibits hydrogen peroxidase induced apoptosis in the cultured neonatal cardiomyocytes. The data show that Shh exerts both direct and indirect effects to preserve myocardial integrity in the setting of acute myocardial infarction.

### EXAMPLE 21

### phShh Induces Myocardial Proliferation

These previous example showed a direct anti-apoptotic effect of Shh on cardiomyocytes. To further document the significance of this effect in vivo, we examined the infarct area in mice treated with Shh or control plasmid. Using a cardiomyocyte specific stain we found that there was a significant difference in the wall thickness within the infarct zone, and that the number of surviving cardiomyocytes was significantly greater in Shh plasmid group (control: 58.8±4.2, phShh: 71.5±8.8/hpf, P<0.05). This finding is important from the standpoint of preservation of functional integrity.

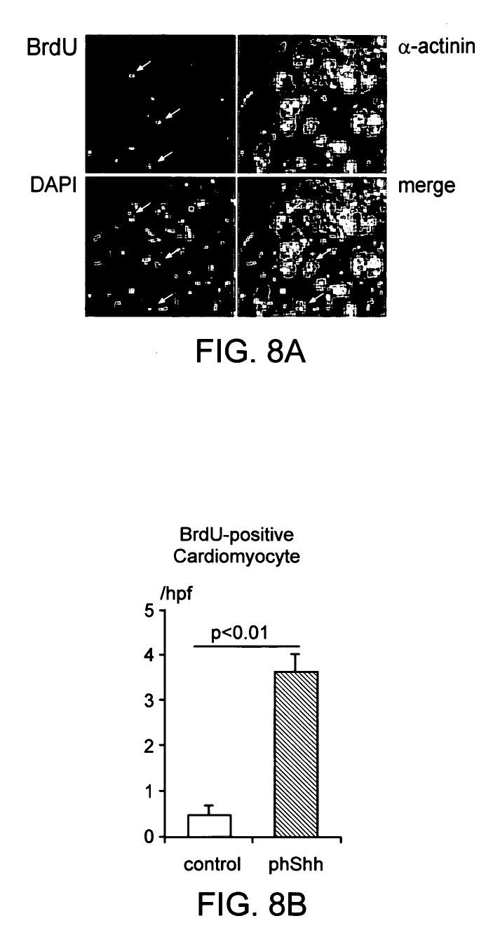

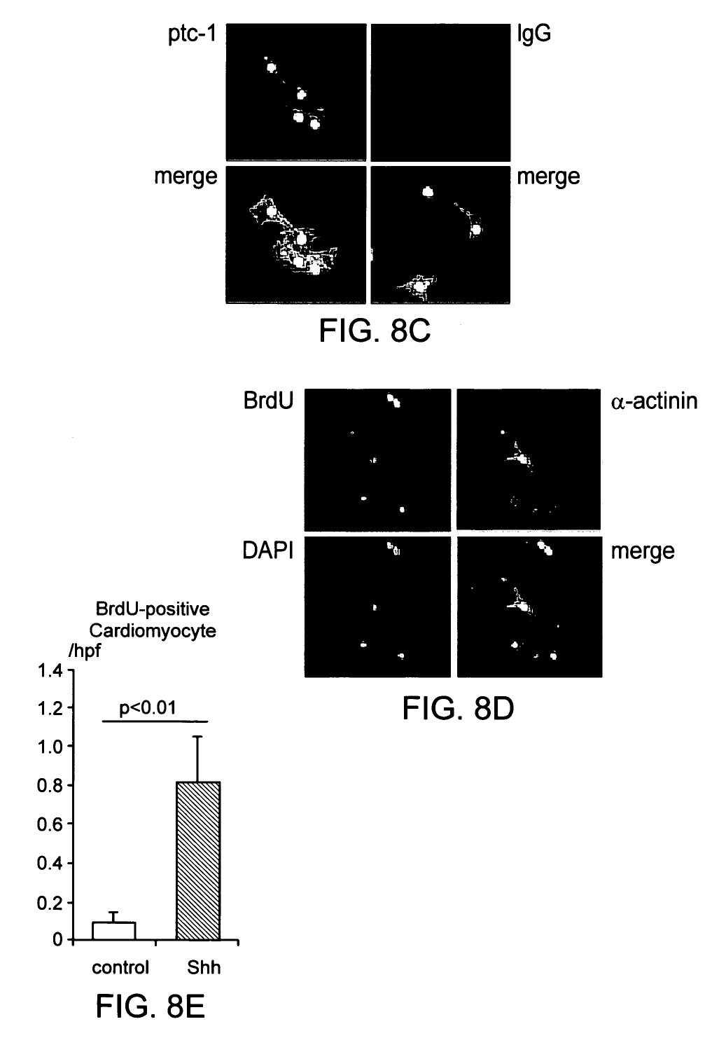

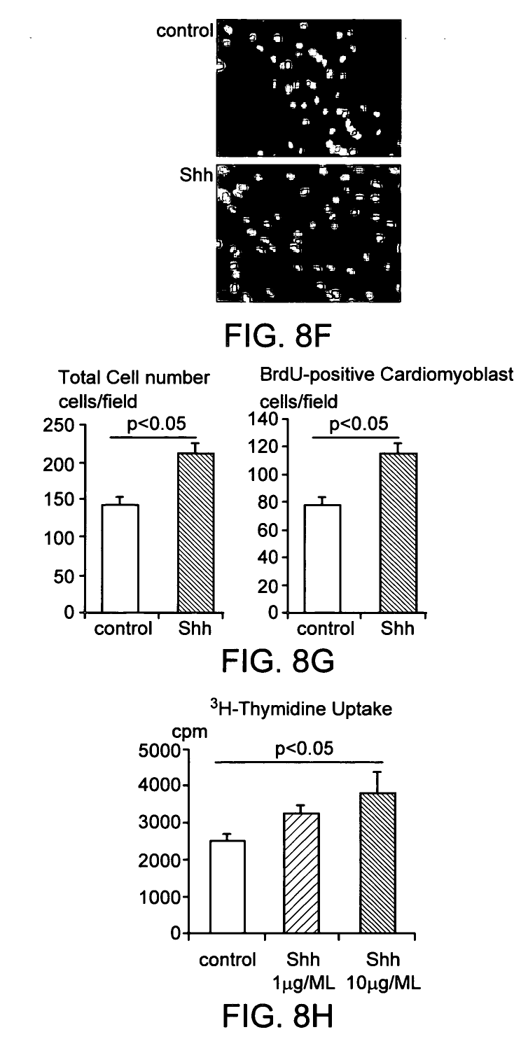

We considered that this finding could be the result of cardiomyocyte protection and/or proliferation. To test the latter possibility, osmotic mini-pumps releasing BrdU continuously for 28 days was implanted into in mice following LAD ligation with Shh or control empty plasmid gene transfer. FIG. 8A shows representative immunostaining for BrdU and α-actinin performed 28 days later. As indicated by the arrows, triple positive cells, expressing α-actinin and revealing incorporation of BrdU, were considered as evidence for cardiomyocyte proliferation. The total number of BrdU positive cardiomyocytes was much greater in the Shh plasmid group (phShh: 3.6±0.5, control: 0.9±0.2/hpf, P<0.01, FIG. 8B).

To verify the proliferative potential of Shh on cardiomyocytes, primary cultured neonatal cardiomyocytes and the H9C2 cardiomyoblast cell line was used for in vitro studies. As shown in FIG. 8C, cardiomyocytes express the cardiac specific marker, α-actinin as well as the Ptc1 receptor both on the cell surface and in the nucleus. Expression of Ptc1 in the nucleas was confirmed Western blotting of nuclear and cytoplasmic fractions of cardiomyocyte cell lysates. 36 h after Shh treatment, more BrdU positive cells were observed in the Shh treated primary cultured neonatal cardiomyocytes (control: 0.08±0.06, Shh: 0.79±0.25/hpf, P<0.01, FIG. 8D,E) and in Shh treated H9C2 cells (control: 62.6±8.9, Shh: 107.3±9.1/hpf, P<0.05, FIG. 8F,G). Furthermore ³H-thymidine uptake was increased in a dose dependent manner by Shh (control: 2587±98, Shh 1 μg/ml: 3312±158, Shh 10 μg/ml: 3892±514 cpm, P<0.05 between control and Shh 10 μg/ml, FIG. 8H). These and other results in the Examples described above (in vivo and in-vitro studies) reveal that Shh exerts a direct proliferative effect on cardiomyocytes.

FIGS. 8A-H are described in further detail as follows: A: representative immunostaining for BrdU, α-actinin and DAPI. Samples were obtained 28 days after induction of myocardial ischemia and implantation of BrdU pumps. Immunofluorescence staining shows a significantly greater number of BrdU (red) positive cells in phShh injected mice at the borderzone of myocardial ischemia. White arrows indicate nuclei (stained blue with DAPI) that co-localize with BrdU (red) in cells that are also positive for alphα-actinin (green), therefore identifying proliferating cardiomyocytes; B: quantification of BrdU positive cardiomyocytes in control (n=7) and phShh group (n=7); C: immunostaining for Ptc1 (red) and α-sarcomeric actinin (green) in primary cultured cardiomyocytes. Shh receptor Ptc1 was observed in both nucleus and cell membrane. Negative control IgG antibody reveals no staining. Co-localization of Ptc1 nuclear immunoreactivity is demonstrated by DAPI counterstain in merged images (separate DAPI stain alone is not shown); D: representative pictures of primary cultured cardiomyocytes (α-actinin positive, green) revealing evidence of proliferation (BrdU incorporation, red); E: quantification of BrdU positive cardiomyocytes in control and Shh treated cardiomyocytes reveals significant increase in cardiomyocyte proliferation induced by Shh. N=6 per group; F: BrdU staining (Pink) of cultured cardiomyoblasts treated with control medium alone or with the addition of Shh. N=6 per group; G: proliferation of cardiomyoblasts (H9C2), was assessed by cell counts and by BrdU incorporation, both revealing a significant increase in proliferative activity in Shh treated cells; H: proliferation of cardiomyoblasts was confirmed by uptake of tritiated thymidine. Recombinant Shh protein increases proliferative activity which is statistically significant at a concentration of 10 μg/ml. N=5 in each experiment.

Thus, the example shows that phShh induced myocardial proliferation. In particular, the data demonstrates that Shh induced cardiomyocyte proliferation. Increased BrdU incorporation in cardiomyocytes within the infarct zone in the mouse model was also seen. It was also learned that GFP positive cells that expressed the myocardial markers, α-actinin, or troponin-I; however, the number of these cells was relatively small (0.20±0.02/hpf, data not shown), compared with the numbers of BrdU positive cardiomyocytes (3.6±0.5/hpf). Thus it was determined that the majority of the BrdU positive cells were not of bone marrow origin.

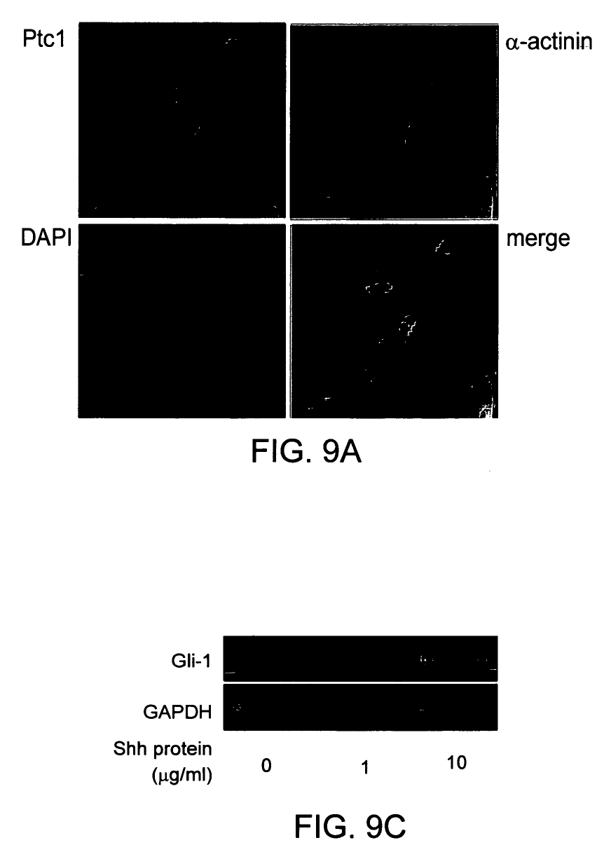

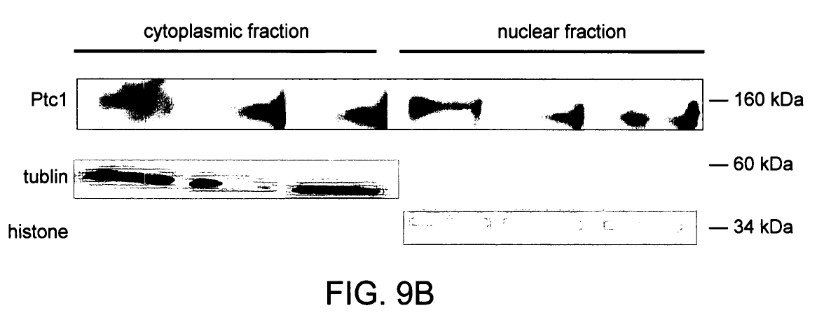

Referring now to figures showing expression of Ptc1 and Gli-1 (FIGS. 9A-C) in cardiomyoblasts+: A: immunostaining of H9C2 demonstrated that Ptc1 was expressed in both the nucleus and cytosol; B. Western blotting of Ptc1 in H9C2 cardiomyoblasts revealed that both fractions of nuclear and cytoplasmic protein expressed Ptc1 protein (N=3 per each group); C: RT-PCR revealed that Shh protein supplementation increases the Hh related transcriptional factor Gli-1.

The following discussion is intended to further enhance appreciation of the invention. It particularly relates to Examples 11-21 above.

The Examples show, among other things, that the Shh pathway can be reconstituted post-naturally in the myocardium for tissue preservation and regeneration. Gene therapy with Shh not only enhances neovascularization but also prevents fibrosis after acute myocardial ischemia. The prevention of replacement fibrosis correlates with the prevention of cardiomyocyte apoptosis, i.e. replacement fibrosis is not required due to the preservation of cardiomyocyte viability. These findings are associated with enhanced cardiac function. Other Shh triggered downstream signaling pathways can also contribute. Bone marrow derived progenitor cells are recruited in increased numbers, associated with an increase in local expression of the trafficking chemokine SDF-1α. Enhanced survival of cardiomyocytes was induced by Shh gene therapy. Without wishing to be bound to theory, this could be a part of the result of upregulation of IGF-1 expression^{23 24}, but in vitro studies also suggest a direct anti-apoptotic effect. Further, Shh induced cardiomyocyte proliferation both in vitro and in vivo.

The result of these effects is evident in the preservation of not only the border zone between infarcted and non ischemic myocardium, but also within the infarct zone itself, where preservation and regeneration of cardiac muscle cells contribute to increased cardiomyocyte density. In the setting of chronic myocardial ischemia, Shh gene therapy also reduces the ischemic area and induces new collateral vessel growth. The Examples showed that the Hh receptor Ptc1 is expressed in the cardiomyocytes, assisting the cytoprotective and proliferative effects documented. Without wishing to be bound to theory, it is believed that multiple favorable mechanisms are associated with a reduction in the extent of LV scarring and enhanced preservation of LV function in the chronic phase after MI. Thus, transient reconstitution of embryonic signaling Shh pathway, encoding for a secretable morphogen, in the peri-infarct period exerts a favorable effects akin to multiple “combined” mono-therapies i.e. angiogenesis, cell therapy, anti-apoptosis and cardiomyocyte proliferation and consistent with Shh role in morphogenesis.

The following material and methods were used as needed to perform the experiments set forth in the Examples.

MI was induced by ligation of coronary arteries (LAD) in 35 male Sprague Dawley rats. Escalating doses of human SHh plasmid (phSHh: 30, 300 and 600 μg), lacZ plasmid (placZ: 300 □1 g) or PBS were injected directly 10 min after LAD ligation. Left ventricular (LV) systolic and diastolic dimensions and fractional shortening assessed by echocardiography were preserved 4 weeks after MI in phSHh-300, 600 μg groups compared with pLacZ group (P<0.01). LV +dP/dt and -dP/dt 4 weeks after MI in phSHh-300, 6000 □g groups were also greater than those in pLacZ group (P<0.05). Capillary density 4 weeks after MI was significantly higher in the phSHh-300, 600 μg groups than pLacZ group (P<0.01). In addition, the ratio of fibrosis area to LV area 4 weeks after MI was also significantly lower in the phSHh −300, 600 μg groups (P<0.01). To evaluate the recruitment of bone-marrow (BM) derived endothelial progenitor cell, we made MI models using 20 FVB mice transplanted with BM from transgenic donors expressing beta-galactosidase transcriptionally regulated by the endothelial cell-specific Tie-2 promotor. Greater numbers of X-gal positive cells were observed at the ischemic sites in the phSHh group both 1 and 4 weeks after MI (P<0.01). RT-PCR showed that SHh upregulated various angiogenic cytokines (VEGF, angiopoietin-1, 2), anti-apoptoic cytokine (IGF) and trafficking chemokine for hematopoietic stem cell (SDF-1α) in both cultured cardiac fibroblasts and MI tissue.

A. Statistical Procedures

All results were expressed as mean±S.E. Statistical significance was evaluated using the unpaired Student's t-test between two means. Multiple comparisons between more than three groups were done by ANOVA. A value of P<0.05 denoted statistical significance

B. Production of Human Shh Plasmid

There are reports that native full length Shh gene product undergoes an auto-processing reaction during its biogenesis resulting in the amino- and carboxy-terminal domain products³⁶. Biologic activity is believed to be within the amino-terminal cleavage product, however, during auto-processing, the amino-terminal domain products were said to be cholesterol modified and this modification causes the amino-terminal protein to be tightly cell associated³⁷, leaving the protein tethered to the cell that made it. Without wishing to be theory bound, it was believed that this would be disadvantageous for a local gene therapy approach. Thus the amino-terminal domain of human Shh coding sequence was selected to make a Shh-plasmid using pCMV-ScriptPCR mammalian expression vector (Stratagene). Human Shh plasmid (phShh) is a 4,878-bp plasmid that contains the 600 bp amino terminal domain coding sequence of human Shh. Expression of Shh gene is modulated by the presence of promoter sequences from cytomegalovirus to drive Shh expression. Downstream from the Shh cDNA is an SV40 polyadenylation sequence. The plasmid also contains a gene that confers neomycin/kanamycin resistance to the host cells.

C. Assessment of Transgene Expression In Vitro

Medium was conditioned by transfected COS cells using liposome based Transfast transfection kit (Promega) according to the manufacturers directions. After 24 h of transfection, the supernatants were harvested and cells extracted with RIPA buffer (50 mM Tris, 150 mM sodium chloride, 1% NP40, 0.5% sodium deoxycholate, 0.1% SDD) with proteinase inhibitors (Roche Molecular Biochemicals) and centrifuged at 3,000 rpm for 20 minutes at 4° C. twice. Total protein extracts were quantified using the BCA protein assay (Pierce, Rockford, Ill.). Total protein extracts (100 μg) were electrophoresed on a 15% sodium dodecyl sulphate (SDS)-polyacrylamide gel and electrophoretically transferred to an Immobilon PVDF membrane (Millipore). Protein standards (BioRad) were run on each gel. The blots were blocked with 5% milk in Tris-buffered saline Tween-20 for 1 hour at room temperature. Blots were incubated overnight at 4° C. with primary antibody, rabbit polyclonal against Shh at 1:500 dilution (Santa Cruz Biotech). After stringent washing, blots were incubated for 1 hour at room temperature with 1:5000 diluted horseradish peroxidase-conjugated anti-rabbit IgG secondary antibody (Santa Cruz Biotech). Peroxidase activity was visualized by exposing an X-ray film to blots incubated with ECL reagent (Amersham).

The conditioned medium from transfected COS cells was also incubated with cultured rat cardiac fibroblasts to examine the expression of the transcriptional factor Gli-1, a downstream target gene of Shh, by RT-PCR as described below.

D. Western Blots

Protein extracts from the hearts of mice, killed 7 days after ischemia were used for Western blot analysis for Ptcl (Santa Cruz Biotech) expression as described previously²⁹. Nuclear and cytoplasmic protein isolation was performed as described previously. In brief, cells were washed 3 times in cold phosphate-buffered saline and then swellen on ice for 20 min in buffer containing 10 mM Tris-HCl (pH 8.0), 1.5 mM MgCl₂ and complete protease inhibitors (Boehringer). After centrifugation at high speed (6000 rpm), the supernatant was collected (cytoplasmic fraction) and the pellet was washed in buffer containing 2 mM Tris-HCl (pH 8.0), 3 mM MgCl₂, 400 mM NaCl, 1 mM EDTA and complete protease inhibitors (Boehringer). After a second high speed centrifugation (13000 rpm), the supernatant was collected (nuclear fraction). Total protein extracts (80 μg) were electrophoresed on a 7.5% SDS-polyacrylamide gel and electrophorefically transferred to an Immobilon PVDF membrane (Millipore). Protein standards (BioRad) were run on each gel. The blots were blocked with 5% milk in Tris-buffered saline Tween-20 for 1 hour at room temperature. Blots were incubated overnight at 4° C. with primary antibody, rabbit polyclonal against Ptc1 at 1:500 dilution (Santa Cruz Biotech). After stringent washing, blots were incubated for 1 hour at room temperature with 1:5000 diluted horseradish peroxidase-conjugated anti-rabbit IgG secondary antibody (Santa Cruz Biotech). Peroxidase activity was visualized by exposing an X-ray film to blots incubated with ECL regent (Amersham). For the loading control, histone (nuclear fraction) and a-tubulin (cytoplasmic fraction) were used.

E. Quantitative RT-PCR

Total RNA was reverse transcribed using iScript cDNA Synthesis Kit (Bio Rad) and amplification was performed on the Taqman 7300 (Applied Biosystems). The PCR conditions were as follows: hold for 2 min at 50° C., and 10 min at 95° C. followed by 2 step PCR for 40 cycles of 95° C. for 15 seconds and 60° C. for 60 seconds. Each sample contained 1 μl cDNA in a 20 μl total reaction using Platinum Quantitative PCR Supermix-UDG (Invitrogen). Primer and probe sequences were as follows:

Shh:
forward 5′-GAGCAGACCGGCTGATGACT-3′,
reverse 5′-AGAGATGGCCAAGGCATTTAAC-3′
and
FAM-AGAGGTGCAAAGACA-MGB.
Dhh:
forward 5′-CGCAGACCGCCTGATGAC-3′,
reverse 5′-GCGATGGCTAGAGCGTTCAC-3′
and
FAM-AGCGTTGCAAAGAG-MGB.
Ihh:
forward 5′-CAAACCGGCTGAGAGCTTTC-3′
reverse 5′-AGCCGACGCGGAGGAT-3′
and
FAM-AGGTCATCGAGACTCA-MGB.
Gli-1:
forward 5′-CGTCACTACCTGGCCTCACA-3′,
reverse 5′-CCCCCTGGCTGAAGCATAT-3′
and
FAM-CCAGCACTACATGCTCCGGGCAA-TAMRA.
VEGF:
forward 5′-CAAAAACGAAAGCGCAAGAAA-3′,
reverse 5′-CGCTCTGAACAAGGCTCACA-3′
and
FAM-CCCGGTTTAAATCCTGGAGCGTTCA-TAMRA.
Angiopoietin-1:
forward 5′-CAGATACAACAGAATGCGGTTCA-3′,
reverse 5′-TGAGACAAGAGGCTGGTTCCTAT-3′
and
FAM-AACCACACGGCCACCATGCTGG-TAMRA.
Angiopoietin-2:
forward 5′-CTACAGGATTCACCTTACAGGACTCA-3′,
reverse 5′-CTTCCTGGTTGGCTGATGCT-3′
and
FAM-TGATTTTGCCCGCCGTGCCT-TAMRA.
IGF-1:
forward 5′-CCTACAAAGTCAGCTCGTTCCA-3′
reverse 5′-TCCTTCTGAGTCTTGGGCATGT-3′
and
FAM-CGGGCCCAGCGCCACACT-TAMRA.
SDF-1α:
forward 5′-ATCAGTTACGGTAAGCCAGTCA-3′,
reverse 5′-TGGCGACATGGCTCTCAAA-3′
and
FAM-CTGAGCTACAGATGCCCCTGCCGATT-TAMRA.

Relative mRNA expression of target genes was calculated with the comparative C_{T} method. The amount of target genes was normalized to the endogenous 18S control gene (Applied Biosystems). Difference in C_{T} values was calculated for each MRNA by taking the mean C_{T} of duplicate reactions and subtracting the mean C_{T} of duplicate reactions for 18S RNA. We calculated the fold change in expression of the target gene from cells treated relative to control cells: relative expression=2^{ΔCT}

F. Experimental Animals

Male C57BL/6J mice (Jackson Laboratories), male or female NLS-Ptc 1-lacZ mice, or their wild type littermates (kindly provided from Dr. Scott, MP at Stanford University) and 6 week old male Sprague-Dawley rats (Charles River Laboratories) were used for acute myocardial ischemia study. We also used bone marrow transplant (BMT) animal models using FVB/N-TgN[TIE2/lacZ] mice as previously described³¹. Tie-2/BMT mice, which receive bone marrow from transgenic mice constitutively overexpressing beta-galactosidase regulated by the endothelial specific Tie-2 promoter. Twenty female FVB mice (4 weeks old) were used as recipients. At 4 weeks after BMT, by which time the BM of the recipient mice is typically reconstituted, BMT mice (at 8 weeks age) underwent ischemia induction by LAD ligation. Seventeen male Yorkshire swine (Pine Acre Rabbitry Farm, Norton, Mass.) weighing 20-25 kg were used for a chronic myocardial ischemia study.

G. Induction of Myocardial Ischemia

All animals were anesthetized with sodium pentobarbital (50 mg/kg IP). Animals were orally intubated with 20GIV (rat) or 22GIV(mice) catheter and artificially ventilated with a respirator (Harvard Apparatus). A small oblique thoracotomy was performed lateral to the midsternal line (rat) or left intercostal line (mice) in the third costal space to expose the heart. After the pericardium was opened, the proximal left anterior descending artery (LAD) branch of the left coronary artery was ligated using 6-0 (rat) or 8-0 (mice) polypropylene sutures through a dissecting microscope.

Seventeen male Yorkshire swine were used for models of chronic myocardial ischemia. Following left thoracotomy, an ameroid constrictor (Research Instruments, SW) was place around the proximal portion of the left circumflex artery (LCX) as described previously⁸.

H. Intramyocardial Gene Delivery

For acute myocardial ischemia (MI), direct myocardial DNA injections were performed 10 minutes after LAD ligation. Rats received an escalating dose of the naked plasmid DNA encoding for human Shh (phShh-30, 300 and 600 μg/0.1 ml sterile saline) subepicardially with a curved 27-gauge needle into 5 sites along the anterior and posterior left ventricular wall. Mice received one dose of phShh (100 μg/0.02 ml sterile saline). Control animals received an equivalent volume of either sterile saline (PBS), lacZ plasmid or empty plasmid. The area of injection corresponded to the region of the myocardium supplied by the left ascending coronary artery (LAD). The chest incision was then closed in layers with 3.0 (rat) or 6-0 (mice) suture.

In the swine study, NOGA nonfluoroscopic LV electroanatomical mapping was performed to guide injections to foci of myocardial ischemia 5 weeks after constrictor placement as described before. In brief, the NOGA system (Johnson & Johnson-Cordis) of catheter-based mapping and navigation has been previously described in detail. Ischemic myocardium was defined as a zone with unipolar voltage (UpV)>automatically determined cut-off, signified by red color in the UpV map and linear local shortening (LLS)<3%. This definition was consistent in all examinations throughout this study. Immediately after the ischemic territory was identified by NOGA mapping, 800 μg of phShh or lacZ plasmid in 3 ml of sterile saline was injected into 6 sites within ischemic myocardium (500 μl to each site) using NOGA injection catheter (MyoSTAR™, Cordis).

I. Physiological Assessment of LV Function

Transthoracic echocardiography (SONOS 5500, Philips Medical Systems, Andover, Mass.) was performed 5, 14 and 28 days after gene delivery in the rat MI model. LV diastolic (LVDd) and systolic (LVDs) dimensions and fractional shortening (FS) were measured at the mid-papillary muscle level. Regional wall motion score was examined per published criteria⁸. An experienced researcher who was blinded to treatment performed all procedures and analyses.

In the swine study, transthoracic echocardiography (SONOS 5500), selective left coronary angiography, and NOGA LV electromechanical mapping were performed 5 weeks after constrictor placement Oust before injection of genes) and 4 weeks after the gene injection. Echocardiographic FS and regional wall motion score were quantified using the LV short axis views at the mid-papillary muscle level. Collateral flow to the LCX territory was graded angiographically in a blinded fashion using the Rentrop scoring system. The area of ischemia was quantified by NOGA mapping as previously described⁸. All data were evaluated by experienced researchers who were blinded to treatment assignment.

J. Hemodynamic Assessment

Cardiac hemodynamics in rats were assessed 28 days after injection of phShh, lacZ plasmid or saline. Rats were anesthetized with sodium pentobarbital (50 mg/kg, IP) and intubated. Body temperature was maintained at 37° C. A 2.0-French high-fidelity Millar pressure catheter (Millar Instruments, Houston) was inserted via the right carotid artery into the left ventricle. After LV function and heart rate stabilized, LV systolic pressure, end-diastolic pressure, developed pressure, maximal positive (+dP/dt) or negative (−dP/dt) pressure development and relatively load-independent systolic function index, dP/dtmax/IP³⁸ (IP: instantaneous pressure) were recorded. Calibration of the Millar catheter was verified before and after each measurement.

K. Immunohistochemical Assessment

28 days after gene delivery, at time of necropsy, rat hearts were sliced into 4 transverse sections from apex to base and fixed with 4% paraformaldehyde. Paraffin-embedded tissues stained by elastic-trichrome were used to measure the average ratio of fibrosis area to left ventricular (LV) area. Immunohistochemical staining was performed using antibodies prepared against the murine-specific endothelial cell marker isolectin B4 (Vector Laboratories) and a-smooth muscle acting (Sigma). Capillary density was evaluated morphometrically by histological examination of 5 randomly selected fields of tissue sections recovered from segments of LV myocardium, subserved by the occluded LAD. Capillaries were recognized as tubular structures positive for isolectin B4. The total number of capillaries in 5 microscope fields from the infarct border zone were counted in 5 tissue sections from each heart. The positive area of α-smooth muscle acting was examined using NIH image. Two examiners who were blinded to treatment performed all morphometric studies.

L. Cellular Identification of LacZ-expressing Cells

Tie-2/lacZ/BMT mice were sacrificed 1 and 4 weeks after MI. NLS-ptc1-lacZ mice were sacrificed 1 week after MI. The explanted hearts were sliced in a bread-loaf fashion into 3-transverse sections from apex to base (base-, mid- and apex-portion). Mid-portions of the hearts were fixed in 4% paraformaldehyde for 3 h at room temperature and incubated in X-gal solution overnight at 37° C. The target tissue samples were then placed in PBS and examined under a dissecting microscope to detect sites of lacZ-expressing cells macroscopically. Also, histological sections were counterstained with nuclear fast red under 40× magnification and counted X-gal positive cells (blue stained cells) per sample in a blinded manner. For β-gal immunofluorescence staining, hearts from Tie2/lacZ/BMT or NLS-Ptc1-lacZ mice were harvested 7 and 28 days after induction of ischemia and frozen or paraffin sections were proceeded. Primary antiserum was rabbit IgG anti-beta-galactosidase (CORTEX Biochem). Goat anti-rabbit IgG biotin conjugated antibody (Signet) was used as secondary antibody. Staining was visualized by using Rhodamine-conjugated streptoavidin (Jackson Lab.) For vimentin and α-actinin immunostaining, heart samples were fixed in 1% paraformaldehyde for 2 hours. The staining was done on frozen sections with anti-vimentin goat serum (Sigma Chemical) or anti-α-actinin goat serum (Sigma Chemical) using FITC conjugated donkey anti-goat antibody. For endothelial cell staining, the staining was done on paraffin sections with the murine-specific EC marker, isolectin B4 (Vector Laboratories).

M. Primary Cultured Adult Cardiac Fibroblasts

To examine the direct effect of Shh on the cardiac fibroblasts, adult cardiac fibroblasts were prepared by selective attachment as described by Crabos and colleagues³⁹. Briefly, adult male Sprague-Dawley rats (200-250 g) were decapitated and cleaned with ethanol before the thorax was incised. Hearts were excised, minced, and washed in PBS (GIBCO) supplemented with antibiotics (penicillin 100 U/ml and streptomycin 100 mg/ml; GIBCO). The tissue was then incubated at 37° C. for 20 min with 0.1% trypsin (Sigma Chemical), 0.1% collagenase (Type CLS, Worthington Biochemicals), and 0.1% BSA (Sigma Chemical). Dissociated cells were centrifuged (for 5 min at 1,000 rpm) at the end of each of several incubation periods, and the cell pellet was resuspended in DMEM (GIBCO) supplemented with 10% fetal bovine serum (FBS; GIBCO) and antibiotics. Cells were then seeded into a 25-cm² flask and incubated at 37° C. in a humidified atmosphere of 5% CO₂-95% air. After 2 h, unattached cells and debris were discarded, and attached cells (nonmuscle cells, mostly fibroblasts) were cultured further in DMEM containing 10% FBS. This procedure yields cultures of cells that are almost exclusively fibroblasts by first passage, as determined by immunocytochemical techniques. For Shh treatments, cells were then passaged with trypsin (0.25%), plated in non-coating 35 mm plates at a density of 8-10,000 cells/cm² in DMEM+10% FBS for 24 h. Oct-Shh protein (hydrophobic modified protein designed to increase its activity⁴⁰ was supplemented at the appropriate concentration (0, 1 and 10 □g/ml) in serum free DMEM+antibiotics supplement. 5 Cells were harvested after 36 h with or without Shh treatment and RNA was extracted using RNA-Stat (Tel-Test Inc.) according to the manufacturer's instructions and quantitative RT-PCR was performed as described above.

N. Detection of Apoptosis

Apoptosis was detected by active-caspase3 staining (Promega) or the TdT-mediated dUTP nick end-labeling (TUTNEL) assay (Roche Molecular Biochemicals) according to the manufacture's description. Mice heart samples were obtained 4 days after LAD ligation. Frozen sections were fixed with 10% formalin for 5 min at room temperature and primary antiserum was rabbit IgG anti-active-caspase3 (Promega). Staining was visualized by using Cy3-conjugated goat anti rabbit antibody. The samples were then incubated with polyclonal antibody against α-actinin for 1 h at 37° C. followed by FITC-conjugated secondary antibody and analyzed by fluorescence microscopy (×400). The positive area of active-caspase3 was analyzed by histological examination from 10 different mice heart sections (N=5 each group) at the ischemic borderzone using NIH image.

To examine anti-apoptoic effect on cardiomyocytes in vitro, neonatal rat cardiomyocytes were prepared from 1-day-old Sprague Dawley rats (Charles River) and cultured as previously described⁴¹. Neonatal cardiomyocytes were seeded into four chamber slides at a density of 10,000/cm² and pretreated with Shh protein (10 μg/ml) for 48 h prior to beginning H₂O₂ (100 μM) treatment in DMEM without serum. It has already reported this dose of H₂O₂ significantly induces myocardial apoptosis in vitro⁴². Following H₂O₂ treatment for 24 h, adherent cells were fixed with 4% paraformaldehyde, and TUNNEL labeling (Roche Molecular Biochemicals) was performed. The samples were then incubated with a monoclonal antibody against a-sarcomeric actinin for 1 h at 37° C. followed by rhodamine conjugated goat anti mouse IgG and analyzed by fluorescence microscopy.

O. Immunofluorescence for Ptc1

The staining was done on PFA fixed cultured cardiomyocytes and H9C2 cardiomyoblasts. Primary antiserum was rabbit IgG anti-patched C-terminus (1:200 dilution, Santa Cruz Biotech) or rabbit IgG for control. Biotinylated anti-rabbit IgG was used as secondary antiserum. Staining was visualized by using rhodamine-conjugated streptavidin (Pharmingen). To confirm the staining, Western blotting of nuclear fraction and cytoplasmic fraction of H9C2 cells was done using the same Ptc1 antibody.

P. Examination of Myocardial Proliferation

To examine the potential of myocardial proliferation, we implanted mini-osmotic pump releasing bromodeoxyuridine (BrdU; 0.473 g/ml in NaHCO₃, 300 μL, Sigma Chemical) continuously for 28 days into 14 mice acute MI with or without phShh injection. After 28 days, mice were sacrificed and heart samples were fixed with 4% PFA for 3 h and cut into 5 μm sections. For BrdU staining, slides were pretreated with microwave 15 min in 10 mM citrate buffer. Then immunofluorescent staining was performed using antibodies prepared against the biotinylated sheep anti-BrdU (BIODESIGN), rabbit polyclonal antibody for α-actinin (Sigma Chemical) and DAPI. The staining was visualized by FITC-conjugated streptoavidin (Jackson Lab.) or Rhodamin-conjugated anti rabbit IgG (Jackson Lab.) Proliferative cardiomyocyte was counted as a triple positive for BrdU, α-actinin and DAPI under fluorescent microscopy (×400) at the ischemic site.

To examine the proliferative potential of Shh on the cultured cardiomyocyte, primary cultured neonatal cardiomyocyte and cardiomyoblast (H9C2) was used. Neonatal cardiomyocytes were prepared as described before⁴¹ and seeded into 24-well plates at a density of 5,000/cm². H9C2 cardiomyoblasts were grown to 40% confluency in 24-well plates. Both cells were followed by serum starvation for 36 h. After this starvation period, medium was aspirated and cells were washed with PBS. Medium containing 10%FBS was then added to the wells and cells were incubated for another 36 h with Shh protein (0 and 10 μg/ml). BrdU (10 μM/well) was added to the cells for the last 15 h. Finally cells were washed, collected and stained with sheep anti-BrdU, mouse anti-α-sarcomeric actinin and DAPI. For BrdU staining, cells were pretreated with 2N-HCl and borate buffer. Proliferative cells were counted as a double positive for BrdU and α-actinin under fluorescent microscopy in randomly selected 6 different fields of each sample (N=6).

Furthermore the effect of Shh protein on the uptake of ³H-thymidine was tested as a measure of cell proliferation. Cardiomyocyte culture usually contaminates cardiac fibroblasts. To avoid the influence of Shh on fibroblast, we used a cardiomyoblast cell line (H9C2). H9C2 cardiomyoblast cells were grown to 40% confluency in 24-well plates, followed by serum starvation for 36 h. After this starvation period, medium was aspirated and cells were washed with PBS. Medium containing 10%FBS was then added to the wells and cells were incubated for another 36 h with Shh protein (0, 1 and 10 μg/ml). ³H-thymidine (3 mCi/well) was added to the cells for the last 15 h. Finally cells were washed, collected and processed to measure incorporated ³H-thymidine a scintillation counter (Beckman, Fullerton, Calif.). Results are expressed as the mean of counts per minute (cpm) in replicate wells. N=5 per group.

In some instances, the foregoing text includes numbered (superscript) citations. The numbers correspond to the references shown below. It is believed that a review of the references will increase appreciation of the present invention.
1. Abraham, W. T. et al. N Engl J Med 346, 1845-53 (2002).
2. Isner, J. M. & Losordo, D. W. Nature Medicine 5, 491-492 (1999).
3. Losordo, D. W. et al. Circulation 105, 2012-8. (2002).
4. Vale, P. R., Losordo, D. & Milliken, C. Circulation 104, II-361 (2001).
5. Vale, P. R. et al. Circulation 103, 2138-43. (2001).
6. Perin, E. C. et al. Circulation 107, 2294-302 (2003).
7. Assmus, B. et al. Circulation 106, 3009-17 (2002).
8. Kawamoto, A. et al. Circulation 107, 461-8 (2003).
9. Takahashi, T., et al. Nature Medicine 5, 434-8 (1999).
10. Yamaguchi, J. et al. Circulation 107, 1322-8 (2003).
11. Nusslein-Volhard C, W. E. Nature 287, 795-801 (1980).
12. Wang, M. et al. Nature Medicine 1, 1184-1188 (1995).
13. Roelink, H. et al. Cell 76, 761-755 (1994).
14. Goodrich, L. & Scott, M. Neuron 21, 1243-1257 (1998).
15. Johnson, R. L. & Tabin, C. J. Cell 90(6), 979-990 (1997).
16. Pepicelli, C. V., Lewis, P. M. & McMahon, A. P. Current Biology 8, 1083-1086 (1998).
17. Ramalho-Santos, M., Melton, D. A. & McMahon, A. P. Development 127, 2763-2772 (2000).
18. St-Jacques, B. et al. Curr Biol 8, 1058-68 (1998).
19. St-Jacques, B., Hammerschmidt, M. & McMahon, A. P. Genes Dev 13, 2072-86 (1999).
20. Pola, R. et al. Nature Medicine 7, 706-711 (2001).
21. Goodrich, L. V., Milenkovic, L., Higgins, K. M. & Scott, M. P. Science 277(5329), 1109-1113 (1997).
22. Gepstein, L. et al. Circulation 98, 2055-2064 (1998).
23. Fujio, Y., Nguyen, T., Wencker, D., Kitsis, R. N. & Walsh, K. Circulation 101, 660-7 (2000).
24. Welch, S. et al. Circ Res 90, 641-8 (2002).
25. Zardoya, R., Abouheif, E. & Meyer, A. Trends Genet 12(12), 496-497 (1996).
26. Bitgood, M. J. & McMahon, . Dev. Biol. 172(1), 126-138 (1995).
27. Kogerman, P. et al. Nature Cell Biology 1(5), 312-319 (1999).
28. Sisson, J. C., Ho, K. S., Suyama, K. & Scott, M. P. Cell 90(2), 235-245 (1997).
29. Pola, R. et al. Circulation 108, 479-85 (2003).
30. Asahara, T. et al. Science 275, 964-967 (1997).
31. Asahara, T. et al. Circ Res 85, 221-8 (1999).
32. Orlic, D. et al. Nature 401, 701-705 (2001).
33. Orlic, D. et al. Proc Natl Acad Sci USA 98, 10344-9. (2001).
34. Beltrami, A. P. et al. Cell 114, 763-76 (2003).
35. Kenney, A. M. & Rowitch, D. H. Mol Cell Biol 20, 9055-67 (2000).
36. Roelink, H. et al. Cell 81(3), 445-455 (1995).
37. Porter, J. A. et al. Cell 86, 21-34 (1996).
38. Kass, D. A. et al. Circulation 76, 1422-36 (1987).
39. Crabos, M., Roth, M., Hahn, A. W. & Erne, P. J Clin Invest 93, 2372-8 (1994).
40. Taylor, F. R. et al. Biochemistry 40, 4359-71 (2001).
41. Aikawa, R. et al. Circulation 102, 2873-9 (2000).
42. Aikawa, R. et al. J Clin Invest 100, 1813-21 (1997).

The invention has been described in detail with reference to preferred embodiments thereof. However, it will be appreciated that those skilled in the art, upon consideration of this disclosure, may make modifications and improvements within the spirit and scope of the invention.

All references disclosed herein are incorporated herein by reference.

1. A method for preventing or treating a myocardial or related disorder in a patient, the method comprising administering to the patient a therapeutically effective amount of a nucleic acid encoding at least one morphogen or an effective fragment thereof, thereby treating the myocardial or related disorder.

2. The method of claim 1, wherein the method further comprises selecting a patient having the disorder and administering the nucleic directly to or near a heart blood vessel in need of treatment.

3. The method of claim 1, wherein the method further comprises expressing the morphogen or fragment thereof in or near-the heart blood vessel of the patient to prevent or treat the myocardial disorder.

4. The method of claim 1, wherein the method further comprises administering the nucleic acid with a stent, catheter, implementation for performing balloon angioplasty, or related device.

5. The method of claim 1, wherein the myocardial tissue has been impacted by one of ischemia, infarction or dysfunction.

6. The method of claim 1, wherein the method further comprises administering to the heart blood vessel of the patient at least one nucleic acid encoding at least one of an angiogenic or hematopoietic protein or effective fragment thereof.

7. The method of claim 1, wherein the method further comprises administering to the mammal an effective amount of at least one morphogenic protein or an effective fragment thereof.

8. The method of claim 1, wherein the method further comprises administering to the patient an effective amount of at least one of an angiogenic or hematopoietic protein.

9. A method for inducing new blood vessel growth in myocardial tissue of a patient having a myocardial or related disorder comprising administering to the patient a therapeutically effective amount of a nucleic acid encoding at least one morphogen; or an effective fragment thereof, thereby inducing new blood vessel growth in the myocardial tissue.

10. The method of claim 9, wherein the method further comprises selecting a patient having the disorder and administering the nucleic to or near a heart blood vessel in need of treatment.

11. The method of claim 9, wherein the method further comprises expressing the morphogen or fragment in or near the heart blood vessel in the patient to prevent or treat the myocardial disorder.

12. The method of claim 9, wherein the method further comprises administering the nucleic acid with a stent, catheter, implementation for performing balloon angioplasty; or related device.

13. The method of claim 9, wherein the myocardial tissue has been impacted by one of ischemia, infarction or dysfunction.

14. The method of claim 9, wherein the method further comprises administering to the heart blood vessel of the patient at least one nucleic acid encoding an angiogenic or hemaotpoietic protein; or effective fragment thereof.

15. The method of claim 9, wherein the method further comprises administering to the patient an effective amount of at least one morphogenic protein or an effective fragment thereof.

16. The method of claim 9, wherein the method further comprises administering to the mammal an effective amount of at least one of an angiogenic or hematopoietic protein.

17. The method of claim 9, wherein the method further comprises administering to the patient a therapeutically effective amount of endothelial cells (ECs) or endothelial cell precursors (EPCs).

18. The method of claim 17, wherein the cells are characterized by having at least one of the following markers: CD34⁺, flk-1⁺, and tie-2⁺.

19. A pharmaceutical product for preventing or treating a myocardial disorder in a patient in need thereof, the product comprising at least one morphogenic protein or effective fragment thereof formulated to be physiologically acceptable to a mammal, the product further comprising means for administering the product to the mammal.

20. The pharmaceutical product of claim 19, wherein the product is sterile and further comprises at least one of an angiogenic or hematopoietic protein or a nucleic acid encoding one of said proteins.

21. The pharmaceutical product of claim 19, further comprising endothelial cells (ECs), endothelial progenitor cells (EPCs) or both cell types.

22. A kit for the introduction of a morphogen into a patient, the kit comprising at least one morphogen or effective fragment thereof and at least one of an angiogenic or hematopoietic protein or a nucleic acid encoding one of said proteins same, the kit further comprising a pharmacologically acceptable carrier solution, means for delivering the morphogen to the patient, and directions for using the kit.

23. The kit of claim 22, wherein the means for delivering the morphogen is a stent, catheter; syringe or related device.

24. A method for preventing or treating a myocardial or related disorder in a patient, the method comprising administering a therapeutically effective amount of a nucleic acid encoding an N-terminal portion of a morphozen selected from the group consisting of human hedgehog (Shh) protein, human desert hedgehog (Dhh) protein and human Indian Hedgehog (Ihh) protein.

25. The method of claim 24, wherein the method further comprises administering the nucleic directly to or near a heart blood vessel in need of treatment.

26. The method of claim 24, wherein the method further comprises expressing the morphogen or fragment directly in t or near the blood vessel in the patient to prevent or treat the myocardial disorder.

27. The method of claim 24, wherein the method further comprises administering the nucleic acid with a stent, catheter, implementation for performing balloon angioplasty, or related device.

28. The method of claim 24, wherein the myocardial tissue has been impacted by one of ischemia, infarction or dysfunction.

29. The method of claim 24, wherein the method further comprises administering to the heart blood vessel of the patient a nucleic acid encoding at least one of an angiogenic or hemaotpoietic protein; or effective fragment thereof.

30. The method of claim 24, wherein the method further comprises administering to the mammal an effective amount of at least one morphogenic protein or an effective fragment thereof.

31. The method of claim 24, wherein the method further comprises administering to the mammal an effective amount of at least one of an angiogenic or hematopoietic protein.

32. A method for inducing new blood vessel growth in myocardial tissue of a patient in need of such treatment, the method comprising administering a therapeutically effective amount of a nucleic acid encoding an N-terminal portion of at least one of human hedgehog (Shh) protein, human desert hedgehog (Dhh) protein and human Indian Hedgehog (Ihh) protein.

33. The method of claim 32, wherein the method further comprises selecting a patient having the disorder and administering the nucleic to or near a heart blood vessel in need of treatment.

34. The method of claim 32, wherein the method further comprises expressing the morphogen or fragment to in or near the blood vessel in the patient to prevent or treat the myocardial disorder.

35. The method of claim 32, wherein the method further comprises administering the nucleic acid with a stent, catheter, implementation for performing balloon angioplasty; or related device.

36. The method of claim 32, wherein the myocardial tissue has been impacted by one of ischemia, infarction or dysfunction.

37. The method of claim 32, wherein the method further comprises administering to the heart blood vessel of the patient a nucleic acid encoding at least one of an angiogenic or hematopoietic protein; or effective fragment thereof.

38. The method of claim 32, wherein the method further comprises administering to the mammal an effective amount of at least one morphogenic protein or an effective fragment thereof.

39. The method of claim 32, wherein the method further comprises administering to the mammal an effective amount of at least one of an angiogenic or hematopoietic protein.

40. The method of claim 32, wherein the method further comprises administering to the patient a therapeutically effective amount of endothelial cells (ECs) or endothelial cell precursors (EPCs).

41. The method of claim 40, wherein the cells are characterized by having at least one of the following markers: CD34⁺, flk-1⁺, and tie-2⁺.

42. A method for increasing cardiomyocyte proliferation, the method comprising contacting cardiomyocytes with an effective amount of an N-terminal portion of at least one morphogen selected from the group consisting of human hedgehog (Shh) protein, human desert hedgehog (Dhh) protein and human Indian Hedgehog (Ihh) protein.

43. The method of claim 42, wherein the method is performed in vitro.

44. The method of claim 42, wherein the method is performed in vivo.

45. A method for increasing production of endothelial cell precursors (EPCs) in a patient, the method comprising administering to the patient a therapeutically effective amount of at least one morphogen selected from the group consisting of an N-terminal portion of human hedgehog (Shh) protein, human desert hedgehog (Dhh) protein and human Indian Hedgehog (Ihh) protein thereby increasing production of the EPCs.

46. The method of claim 45, wherein the method further comprises increasing neovascularization in the patient.

47. A method for increasing production of a cytokine in a patient, the method comprising administering a therapeutically effective amount of at least one morphogen selected from the group consisting of an N-terminal portion of human hedgehog (Shh) protein, human desert hedgehog (Dhh) protein and human Indian Hedgehog (Ihh) protein to the patient thereby increasing production of the cytokine.

48. The method of claim 47, wherein the cytokine is SDF-1 alpha, vascular endothelial growth factor (VEGF), angiopoietin-1, angiopoietin-2, or IGF-1.

49. The method of claim 1, wherein the morphogen is Sonic hedgehog (SHh), Desert hedgehog (DHh) or Indian hedgehog (IHh).
